(19) United States
(12) Patent Application Publication
(10) Pub. No.: US 2025/0155727 A1
(43) Pub. Date: May 15, 2025

(54) Methods and Devices for Chromatic Aberration Correction

(71) Applicant: Clerio Vision, Inc., Rochester, NY (US)

(72) Inventors: Gustavo A. Gandara-Montano, Rochester, NY (US); Leonard Zheleznyak, Pittsford, NY (US)

(73) Assignee: Clerio Vision, Inc., Rochester, NY (US)

(21) Appl. No.: 19/019,984

(22) Filed: Jan. 14, 2025

Related U.S. Application Data
(63) Continuation of application No. PCT/US2023/028115, filed on Jul. 19, 2023.
(60) Provisional application No. 63/390,700, filed on Jul. 20, 2022.

(51) Int. Cl. G02C 7/02

(52) U.S. Cl. CPC G02C 7/02; G02C 2202/22

(57) ABSTRACT

Chromatic aberrations correcting optical structures provide a chromatic aberration wavefront correction for an optical component or system. A chromatic aberration correcting lens includes at least one subsurface diffractive optical structure. Each of sub-volumes of the at least one subsurface diffractive optical structure has a respective refractive index spatial distribution that differs from a lens material refractive index of the lens. The at least one subsurface diffractive optical structure is configured to induce a chromatic aberration correction for the optical component or system.

### CROSS REFERENCES TO RELATED APPLICATIONS

This application is a Continuation of PCT/US2023/028115 filed Jul. 19, 2023; which claims priority to U.S. Provisional Patent Application No. 63/390,700 filed Jul. 20, 2022, which are incorporated herein by reference in their entirety and for all purposes.

### BACKGROUND

Chromatic aberrations can be generated by a lens made from a single material. As a result of the refractive index of a single material lens being inversely proportional to the wavelength of light passing through the lens, the various wavelengths of light in white light passing through such a lens are refracted in different directions, thereby causing the lens to focus the different wavelengths of light to different points. The refractive index of a single material lens for blue light (400 to 500 nm wavelength) is greater than the refractive index of the same single material lens for red light (620 to 750 nm wavelength) due to the shorter wavelength of blue light relative to red light. Focusing different wavelengths of light to different points typically degrades image quality. Moreover, the human eye generates longitudinal chromatic aberration that reportedly averages approximately 1.75 D between 420 and 660 nm.

### BRIEF SUMMARY

The following presents a simplified summary of some embodiments of the invention to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Embodiments described herein are directed to lenses configured to correct a chromatic aberration and methods of configuring and producing the lenses. The chromatic aberration correcting lenses described herein include at least one subsurface diffractive optical structure formed by inducing localized subsurface changes of the refractive index of the lens. In many embodiments, a chromatic aberration correcting lens has an external shape configured to provide a first non-zero optical power and at least one subsurface diffractive optical structure configured to provide a second non-zero optical power so that the combination of the first non-zero optical power and the second non-zero optical power causes the lens to provide a desired optical power correction. In some embodiments, the at least one subsurface diffractive optical structure provides a chromatic aberration correction without providing an optical power correction for a reference (e.g., a central or design) wavelength for the lens. In some embodiments, a chromatic aberration correcting lens has an external shape configured to provide no optical correction and at least one subsurface diffractive optical structure configured to correct a chromatic aberration and, in some embodiments, further configured to provide a desired optical correction for the central or design wavelength for the lens. Since the chromatic aberration correcting lens configurations described herein can employ a smooth exterior surface, the chromatic aberration correcting lens configurations can be used for any suitable ophthalmic lens (e.g., contact lens, intra-ocular lens), a cornea, a natural lens of an eye, and any suitable non-ophthalmic lens. As described herein, correction of chromatic aberrations can result in improved image quality.

Thus, in one aspect, a lens includes an external surface shape and at least one subsurface diffractive optical structure. The external surface shape is configured to alter a wavefront via refraction. Each of the sub-volumes of the at least one subsurface diffractive optical structure has a respective refractive index spatial distribution that differs from a lens material refractive index of the lens. The at least one subsurface diffractive optical structure and the external surface shape are configured to jointly provide a chromatic aberration correction.

The lens can employ any suitable combination of features described herein. For example, the at least one subsurface diffractive optical structure can be configured to provide a positive diopter correction, zero diopter correction, or a negative diopter correction. The chromatic aberration correction can be configured to correct a native chromatic aberration of an eye. The at least one subsurface diffractive optical structure and the external surface shape can be configured to jointly provide an optical power correction for the eye.

In some embodiments, the at least one subsurface diffractive optical structure includes a subsurface chromatic aberration correcting structure configured to contribute to the chromatic aberration correction and provide zero optical power correction for a reference wavelength of light. For example, each of the sub-volumes of the subsurface chromatic aberration correcting structure can have a respective refractive index spatial distribution so that the subsurface chromatic aberration correcting structure provides a reference wavefront correction comprising optical phase discontinuity regions and piston regions of different constant whole number optical phase in waves with respect to the reference wavelength of light. Each of the optical phase discontinuity regions can extend between and separates respective two immediately adjacent instances of the piston regions. The subsurface chromatic aberration correcting structure can be configured to provide a respective chromatic aberration correction for wavelengths that differ from the reference wavelength of light. The subsurface chromatic aberration correcting structure can be configured to provide no chromatic aberration correction for the reference wavelength of light. The piston regions can include a central piston region and annular piston regions that surround the central piston region. Each of the central piston region and the annular piston regions can be configured to induce a different constant whole number optical phase change in waves with respect to the reference wavelength of light. In some embodiments, the annular piston regions include three of the annular piston regions. In some embodiments, the annular piston regions include six of the annular piston regions. In some embodiments, the annular piston regions include nine of the annular piston regions. In some embodiments, each of the optical phase discontinuity regions provides a one optical wave discontinuity with respect to the reference wavelength of light. In some embodiments, the central piston region is configured to induce no changes in waves with respect to the reference wavelength of light. In many embodiments, the lens includes a lens body made of a transparent material and the subsurface chromatic aberration correcting structure includes portions of the lens body that have a distribution of varying refractive index.

The chromatic aberration correcting lens can be configured to only provide a chromatic aberration correction. For example, the at least one subsurface diffractive optical structure and the external surface shape can be configured to combine to provide no optical power correction for the eye for a reference (e.g., design) wavelength.

The chromatic aberration correcting lens can be configured as any suitable ophthalmic lens. For example, the chromatic aberration correcting lens can be configured as a contact lens, an intraocular lens, or a spectacle lens.

The chromatic aberration correcting lens can be configured to provide any suitable amount of chromatic aberration correction. For example, the chromatic aberration correcting lens can be configured to result in the best-focus Strehl Ratio value occurring at the same dioptric power for all the visible light wavelengths.

In another aspect, a lens includes a subsurface chromatic aberration correcting optical structure. Each sub-volume of the subsurface chromatic aberration correcting optical structure has a respective refractive index spatial distribution so that the subsurface chromatic aberration correcting optical structure provides a reference wavefront correction comprising piston regions of different constant whole number optical phase in waves with respect to a reference wavelength and optical phase discontinuity regions. Each of the optical phase discontinuity regions extends between and separates respective two immediately adjacent instances of the piston regions. The subsurface chromatic aberration correcting optical structure provides a respective chromatic aberration correction for wavelengths that differ from the reference wavelength. In many embodiments, the subsurface chromatic aberration correcting optical structure provides no chromatic aberration correction for the reference wavelength.

The lens can have any suitable configuration. For example, the lens can be configured as an ophthalmic lens (e.g., a contact lens, an intraocular lens, a spectacle lens).

In many embodiments, the lens provides improved image quality for at least one wavelength that differs from the reference wavelength. For example, in some embodiments, the lens is configured to produce substantially the same best-focus Strehl Ratio value for an eye at the reference wavelength while yielding an improved Strehl Ratio at the same focus for a second reference wavelength that differs from the reference wavelength by at least 50 nm. In some embodiments, the lens is configured to result in all visible light wavelengths having the same dioptric power (i.e., all the visible light wavelengths having the same best focus) for an eye, while producing substantially the same best-focus Strehl Ratio value for the eye at the reference wavelength. In some embodiments, the lens is further configured to provide an optical power correction for an eye.

In many embodiments, the piston regions include a central piston region and annular piston regions that surround the central piston region. In many embodiments, each of the central piston region and the annular piston regions is configured to have a different constant whole number of optical phase in waves with respect to the reference wavelength.

The subsurface chromatic aberration correcting optical structure can include any suitable number of the annular piston regions. For example, in many embodiments, the subsurface chromatic aberration correcting optical structure includes three of the annular piston regions. In some embodiments the subsurface chromatic aberration correcting optical structure includes six of the annular piston regions. In some embodiments, the subsurface chromatic aberration correcting optical structure includes nine of the annular piston regions.

Each of the optical phase discontinuity regions can provide any suitable whole number of optical waves of discontinuity with respect to the reference wavelength. In many embodiments, each of the optical phase discontinuity regions provides a one optical wave discontinuity with respect to the reference wavelength. In many embodiments, the central piston region provides zero waves with respect to the reference wavelength.

In many embodiments, the lens includes a lens body made of a transparent material. In many embodiments, the subsurface chromatic aberration correcting optical structure includes portions of the lens body that have a distribution of varying refractive index.

In another aspect, a method of modifying a lens to provide a chromatic aberration correction includes inducing changes in refractive index within the lens to form a subsurface chromatic aberration correcting optical structure. Each sub-volume of the subsurface chromatic aberration correcting optical structure has a respective refractive index spatial distribution so that the subsurface chromatic aberration correcting optical structure provides a reference wavefront correction comprising piston regions of different constant whole number optical phase in waves with respect to a reference wavelength and optical phase discontinuity regions. Each of the optical phase discontinuity regions extends between and separates respective two immediately adjacent instances of the piston regions. The subsurface chromatic aberration correcting optical structure provides a respective chromatic aberration correction for wavelengths that differ from the reference wavelength. In many embodiments, the subsurface chromatic aberration correcting optical structure provides no chromatic aberration correction for the reference wavelength. In many embodiments, inducing the changes in refractive index within the lens to form the subsurface chromatic aberration correcting optical structure comprises directing one or more energy beams into the lens.

The method of modifying a lens to provide a chromatic aberration correction can further include any suitable additional act used to define the changes in refractive index in the lens. For example, the method can further include determining a variation in optical power with wavelength for the chromatic aberration correction. The method can further include determining an equivalent optical power for the subsurface chromatic aberration correcting optical structure. The method can further include generating an optical phase diagram for the subsurface chromatic aberration correcting optical structure. The method can include determining a magnitude for each of the changes in refractive index within the lens.

The method of modifying a lens to provide a chromatic aberration correction can be practiced with any suitable lens. For example, the lens can be configured as an ophthalmic lens (e.g., a contact lens, an intraocular lens, a spectacle lens). The lens can be a natural lens (e.g., a cornea, a natural crystalline lens).

In many embodiments of the method, the lens provides improved image quality for at least one wavelength that differs from the reference wavelength. For example, in some embodiments of the method, the lens is configured to produce substantially the same best-focus Strehl Ratio value for an eye at the reference wavelength while yielding an improved Strehl Ratio at the same focus for a second reference wavelength that differs from the reference wavelength by at least 50 nm. In some embodiments, the lens is configured to result in all visible light wavelengths having the same dioptric power (i.e., all the visible light wavelengths having the same best focus) for an eye, while producing substantially the same best-focus Strehl Ratio value for the eye at the reference wavelength. In some embodiments of the method, the lens is further configured to provide an optical power correction for an eye.

In many embodiments of the method, the piston regions include a central piston region and annular piston regions that surround the central piston region. In many embodiments of the method, each of the central piston region and the annular piston regions is configured to have a different constant whole number of optical phase in waves with respect to the reference wavelength.

In many embodiments of the method, the subsurface chromatic aberration correcting optical structure includes three of the annular piston regions. In some embodiments of the method, the subsurface chromatic aberration correcting optical structure includes six of the annular piston regions. In some embodiments of the method, the subsurface chromatic aberration correcting optical structure includes nine of the annular piston regions.

In many embodiments of the method, each of the optical phase discontinuity regions provides a suitable whole number of optical waves of discontinuity with respect to the reference wavelength. In many embodiments of the method, each of the optical phase discontinuity regions provides a one optical wave discontinuity with respect to the reference wavelength. In many embodiments of the method, the central piston region provides zero waves with respect to the reference wavelength.

For a fuller understanding of the nature and advantages of the present invention, reference should be made to the ensuing detailed description and accompanying drawings.

### BRIEF DESCRIPTION OF THE DRAWINGS

FIG. 1 illustrates longitudinal chromatic aberrations generated by a lens.

FIG. 2 illustrates the chromatic aberration (variation in optical power with wavelength) of an example myopic eye.

FIG. 3 illustrates the chromatic aberration (variation in optical power with wavelength) of a monofocal diffractive contact lens for the example myopic eye.

FIG. 4 illustrates the chromatic aberration (variation in optical power with wavelength) of the combination of the example myopic eye of FIG. 2 and the monofocal diffractive contact lens of FIG. 3.

FIG. 5 shows a plot of Strehl Ratio versus focus for the corrected example myopic eye of FIG. 4 for each of three different wavelengths.

FIG. 6 shows plots of variation in optical power with wavelength for different lenses with different external shapes configured to provide different optical power corrections.

FIG. 7 shows plots of variation in optical power with wavelength for different subsurface diffractive optical structures configured to provide different optical power corrections.

FIG. 8 shows a plot of optical phase in waves (for a reference wavelength of 550 nm) versus radial coordinate for an example lens that induces a chromatic aberration.

FIG. 9 shows a plot of optical phase in waves (for a reference wavelength of 550 nm) versus radial coordinate for a phase-wrapped lens equivalent to the example lens of FIG. 8, using a phase-wrapping value of 1 wave.

FIG. 10 shows an example chromatic aberration correcting lens configured to correct a chromatic aberration of an optical component or an optical system, in accordance with embodiments.

FIG. 11 show a simplified block diagram of a method of configuring a lens for correcting a chromatic aberration, in accordance with embodiments.

FIG. 12 shows a plot of Strehl Ratio versus focus for each of three different wavelengths for the corrected example myopic eye of FIG. 4 combined with the chromatic aberration correcting lens of FIG. 10.

FIG. 13 shows another example chromatic aberration correcting lens configured for correcting a chromatic aberration of an optical component or an optical system, in accordance with embodiments.

FIG. 14 shows a plot of optical phase in waves (for a reference wavelength of 550 nm) versus radial coordinate for an example chromatic aberration correcting subsurface diffractive optical structure, in accordance with embodiments.

FIG. 15 shows a plot of optical phase in waves versus radial coordinate for another chromatic aberration correcting subsurface diffractive optical structure, in accordance with embodiments.

FIG. 16 show a simplified block diagram of a method of configuring a lens for correcting a chromatic aberration, in accordance with embodiments.

FIG. 17 shows another example chromatic aberration correcting lens configured for correcting a chromatic aberration of an optical component or an optical system, in accordance with embodiments.

FIG. 18 shows plots of the maximum diameter of a chromatic aberration correcting subsurface diffractive optical structure that can be generated with a maximum phase available for several examples of dioptric powers of phase-wrapped optical structures that need to be subtracted from the corresponding continuous phase optical structure of the same dioptric power to generate the desired chromatic aberration correcting subsurface diffractive optical structure.

FIG. 19 shows a plot of the maximum phase necessary to create a 6 mm diameter chromatic aberration correcting subsurface diffractive optical structure as a function of the dioptric power of the phase-wrapped optical structure that needs to be subtracted from the continuous phase optical structure with the same dioptric power that generates the desired chromatic aberration correcting subsurface diffractive optical structure.

FIG. 20 is a schematic representation of a system that can be used to form subsurface optical elements within a lens, in accordance with embodiments.

FIG. 21 and FIG. 22 schematically illustrate another system that can be used to form subsurface optical elements within a lens, in accordance with embodiments.

FIG. 23 illustrates an example radial distribution of an optical correction for implementation via subsurface optical elements formed within a lens, in accordance with embodiments.

FIG. 24 illustrates a 1-wave phase wrapped distribution for the example optical correction of FIG. 23.

FIG. 25 illustrates a ⅓ wave ratio of the 1-wave phase wrapped distribution of FIG. 24.

FIG. 26 graphically illustrates diffraction efficiency for near focus and far focus versus optical phase height.

FIG. 27 graphically illustrates an example calibration curve for resulting optical phase change height as a function of laser pulse train optical power.

FIG. 28 is a plan view illustration of a lens that includes subsurface optical structures, in accordance with embodiments.

FIG. 29 is a plan view illustration of subsurface optical structures of the lens of FIG. 28.

FIG. 30 is a side view illustration of the subsurface optical structures of the lens of FIG. 29.

### DETAILED DESCRIPTION

In the description herein, various embodiments are described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

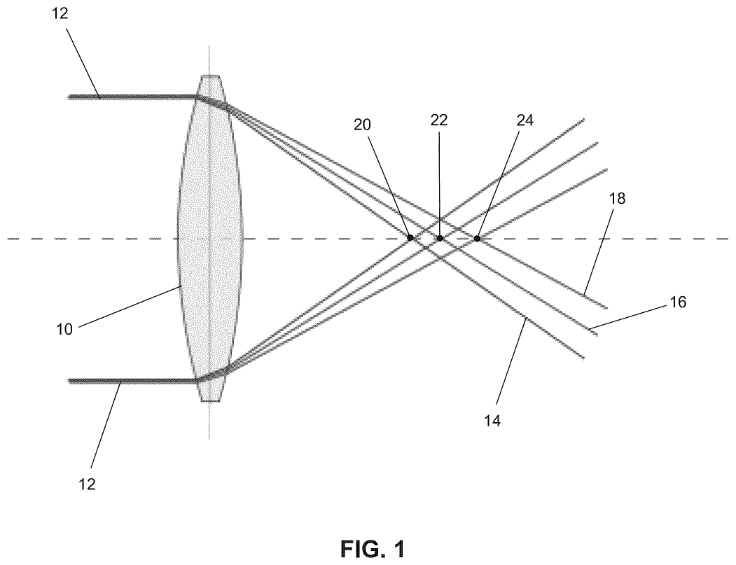

Turning now to the drawing figures in which similar reference numbers refer to similar features in the various drawing figures, FIG. 1 illustrates longitudinal chromatic aberrations generated by an example convex lens 10. Incoming parallel white light 12, which includes a blue light wavelength 14 (e.g., 450 nm), a green light wavelength 16 (e.g., 550 nm), and a red-light wavelength 18 (e.g., 650 nm), is converged by the convex lens 10. Due to the refractive index of the lens 10 being inversely proportional to wavelength, the blue light wavelength 14 is converged more than the green light wavelength 16, which is converged more than the red-light wavelength 18. As a result, the blue light wavelength 14, the green light wavelength 16, and the red-light wavelength 18 are focused to different focal points 20, 22, 24. Focusing different wavelengths of light to different points degrades image quality.

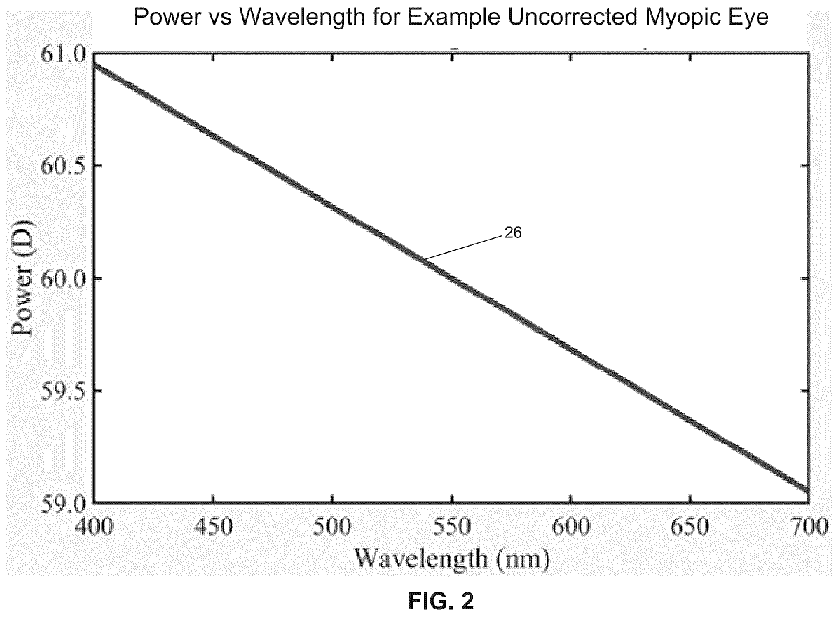

Chromatic aberrations are present in many existing optical systems. For example, FIG. 2 shows a plot 26 of variation in optical power with wavelength of an example uncorrected myopic eye (relative to the central wavelength). At the center wavelength (550 nm), the example uncorrected myopic eye provides 60.0 diopters of optical power. At 400 nm and 700 nm, the example uncorrected myopic eye provides 60.95 and 59.05 diopters of optical power, respectively.

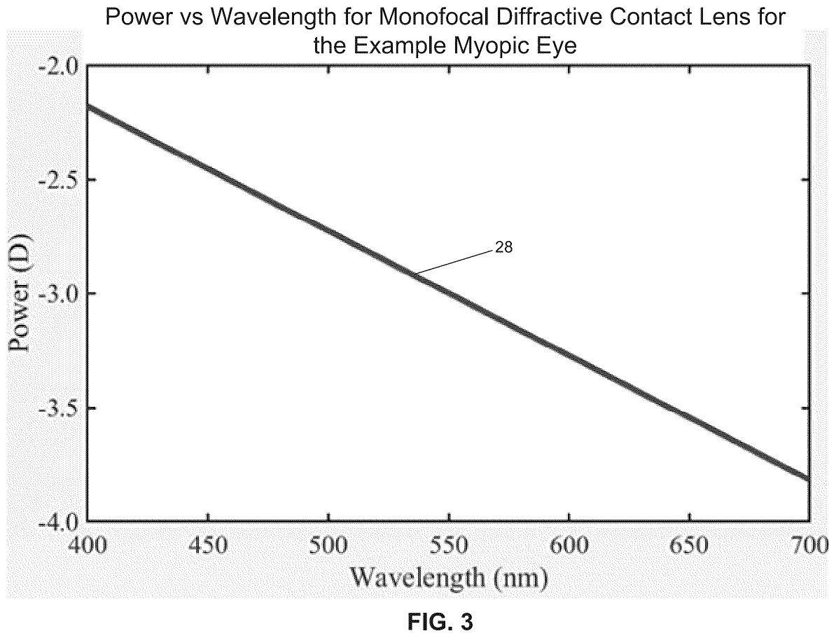

FIG. 3 shows a plot 28 of variation in optical power with wavelength of an example contact lens with a subsurface monofocal diffractive optical structure that can be employed to correct the example myopic eye. At the center wavelength (550 nm), the example contact lens provides −3.0 diopters of optical power. At 400 nm and 700 nm, the example contact lens provides −2.18 and −3.82 diopters of optical power, respectively.

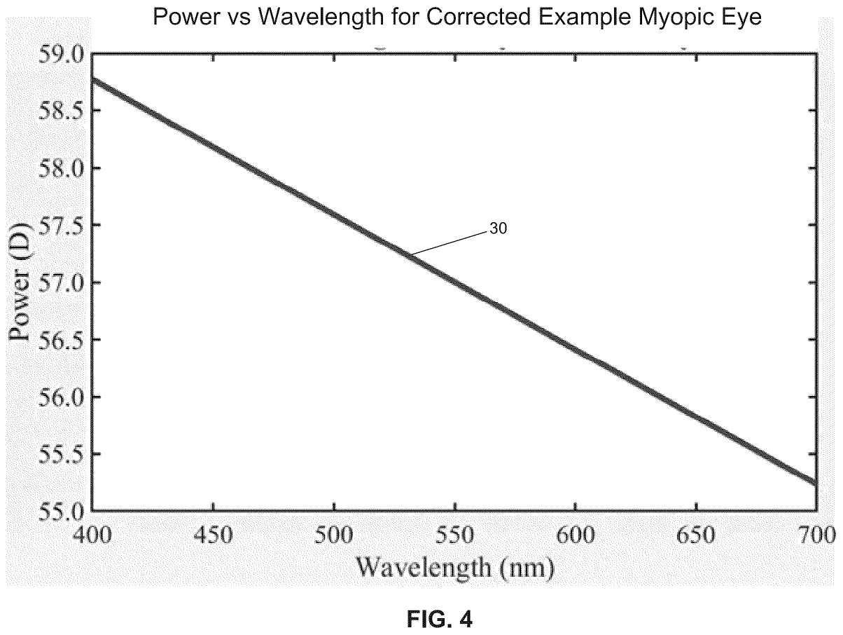

FIG. 4 shows a plot 30 of variation in optical power with wavelength of the combination of the example uncorrected myopic eye and the example contact lens. At the center wavelength (550 nm), the resulting corrected example eye provides −57.0 diopters of optical power. At 400 nm and 700 nm, the corrected example eye provides 58.77 and 55.23 diopters of optical power, respectively.

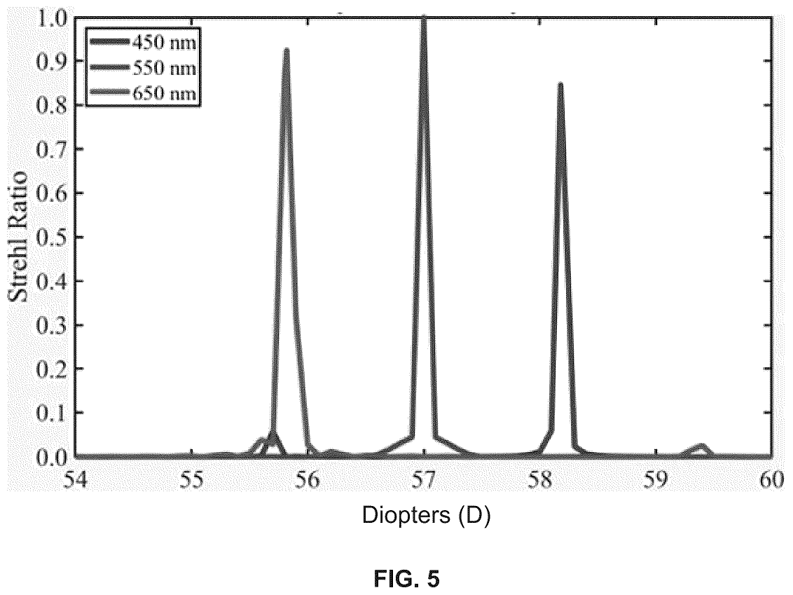

As a result of the variation of optical power of the example corrected eye with wavelength, the image quality provided by example corrected eye is suboptimal. FIG. 5 shows a plot of Strehl Ratio as a function of dioptric power for each of the three different wavelengths (450, 550, and 650 nm) for the example corrected eye of FIG. 4.

### Achromatization

The magnitude of the chromatic aberration induced by an optical component or system can be quantified by the change in optical power (e.g., in diopters) divided by the change in wavelength (e.g., in nm). For an optical system, the total magnitude of chromatic aberration is equal to the sum of the chromatic aberrations induced by the constituent parts of the optical system.

In many embodiments, a chromatic aberration correcting lens is configured for use with an optical system to achromatize the optical system. In many embodiments, a chromatic aberration correcting lens includes a subsurface diffractive optical structure that induces a counter-acting chromatic aberration. The subsurface diffractive optical structure can further be configured to provide a desired optical power. The chromatic aberration correcting lens may further have an exterior shape configured to provide an optical power and induce a chromatic aberration (which can be counter acting or aggravating).

In the following example, a first example chromatic aberration correcting lens is configured as a contact lens for counteracting the chromatic aberration of an example corrected eye of FIG. 4. The first example chromatic aberration correcting contact lens includes a subsurface diffractive optical structure that provides an optical power at the central wavelength. The first example chromatic aberration correcting contact lens also has an exterior shape configured to provide an optical power and induce a chromatic aberration.

Equation (1) and equation (2) can be used to calculate the resulting residual chromatic aberration of an eye as the sum of the native chromatic aberration of the eye and the chromatic aberration induced by the contact lens.

$${( \frac{Δ P}{Δλ} )}_{System} = {( \frac{Δ P}{Δλ} )}_{Eye} + {( \frac{Δ P}{Δλ} )}_{Contact} \qquad Equation ( 1 )$$

$${( \frac{Δ P}{Δλ} )}_{Contact} = {( \frac{Δ P}{Δλ} )}_{Contact lens shape} + {( \frac{Δ P}{Δλ} )}_{subsurface structure} \\ wherein \\ ( \frac{Δ P}{Δλ} ) = change in optical power per change in wavelength ; \\ {( \frac{Δ P}{Δλ} )}_{System} = resulting residual chromatic \\ aberration of the corrected eye ; \\ {( \frac{Δ P}{Δλ} )}_{Eye} = native chromatic aberration of the eye ; \\ {( \frac{Δ P}{Δλ} )}_{Contact lens shape} = chromatic aberration induced \\ by the contact lens shape ; \\ {( \frac{Δ P}{Δλ} )}_{subsurface structure} = chromatic aberration \\ induced by the contact lens subsurface optical structure . \qquad Equation ( 2 )$$

Equation (3) can be used to estimate the native chromatic aberration of the eye.

$${( \frac{Δ P}{Δλ} )}_{eye} = ( {P}_{d , eye} ) ( \frac{1}{{v}_{water}} ) ( \frac{1}{{λ}_{F} - {λ}_{C}} ) \qquad Equation ( 3 )$$

wherein:
Equation 3 is premised on v_{eye}≅v_{water}
(P_{d,eye})=optical power of the eye at the central wavelength;
v_{water}=Abbe number of water (approximation of the Abbe number of the eye)
λ_{F}=486.1 nm (blue Fraunhofer F line from hydrogen)
λ_{C}=656.3 nm (red Fraunhofer C line from hydrogen)

Equation (4) can be used to estimate the chromatic aberration induced by the contact lens shape.

$${( \frac{Δ P}{Δλ} )}_{contact lens shape} = ( {P}_{d , contact lens shape} ) ( \frac{1}{{v}_{water}} ) ( \frac{1}{{λ}_{F} - {λ}_{C}} ) \qquad Equation ( 4 )$$

wherein:
Equation 4 is premised on v_{contact}≅v_{water}
(P_{d,contac lens shape})=optical power provided by the contact lens shape at the central wavelength;
v_{water}=Abbe number of water (approximation of the Abbe number of the contact)
λ_{F}=486.1 nm (blue Fraunhofer F line from hydrogen)
λ_{C}=656.3 nm (red Fraunhofer C line from hydrogen)

Equation (5) can be used to estimate the chromatic aberration induced by the contact lens subsurface diffractive optical structure.

$${( \frac{Δ P}{Δλ} )}_{subsurface structure} = \frac{{P}_{subsurface structure at central wavelength}}{{λ}_{central}} \qquad Equation ( 5 )$$

wherein:
(P_{subsurface structure at central wavelength})=optical power provided by the subsurface structure at the central wavelength;
λ_{central}=the central wavelength

Setting the resulting residual chromatic aberration of the corrected eye to zero in equation (1) results in two remaining variables to be solved for (i.e., the optical power (at the central wavelength) provided by the contact lens shape and the optical power (at the central wavelength) provided by the subsurface optical structure). Since the total optical power provided by the contact lens is equal to the sum of the optical power provided by the lens shape and the optical power provided by the subsurface diffractive optical structure, the desired total resulting power of the contact lens for a given eye provides a second equation that relates the power provided by the contact lens shape and the power provided by the subsurface diffractive optical structure. For the example myopic eye of FIG. 2 for which the desired total resulting power of the contact lens is −3.0 diopters, the optical power provided by the contact lens shape added to the optical power provided by the subsurface diffractive optical structure is equal to −3.0 diopters. As a result, the combination of equation (1) and knowing the desired total resulting power of the contact lens provides two equations and two unknowns (i.e., the optical power provided by the contact lens shape and the optical power provided by the subsurface diffractive optical structure).

For the example myopic eye of FIG. 2, the chromatic aberration can be obtained directly from FIG. 2. At 400 nm, the optical power of the example myopic eye is 60.95 diopters. At 700 nm, the optical power of the example myopic eye is 59.05. Therefore, the chromatic aberration of the example myopic eye (i.e., change in optical power per change in wavelength) is:

$${( \frac{Δ P}{Δλ} )}_{Eye} = \frac{59.05 diopters - 60.95 diopters}{700 nm - 400 nm} = - 0.0063 \frac{diopters}{nm}$$

The chromatic aberration of the example myopic eye of FIG. 2 can also be calculated using equation (3).

$${( \frac{Δ P}{Δλ} )}_{eye} = ( {P}_{d , eye} ) ( \frac{1}{{v}_{water}} ) ( \frac{1}{{λ}_{F} - {λ}_{C}} ) \\ {( \frac{Δ P}{Δλ} )}_{eye} = ( 60 diopters ) ( \frac{1}{55.74} ) ( \frac{1}{486.1 nm - 656.3 nm} ) = - \text{.0063} \frac{diopters}{nm}$$

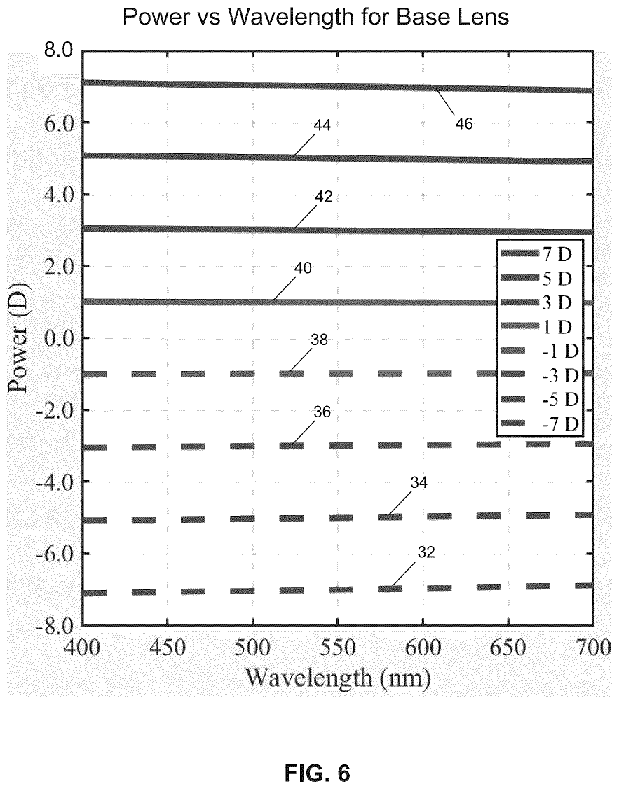

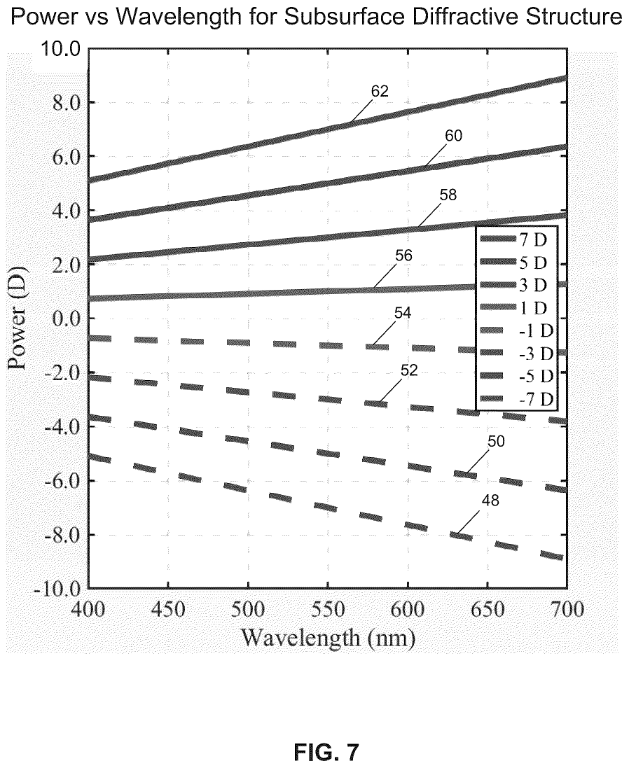

To achromatize and correct the example myopic eye, the first example chromatic aberration correcting contact lens can be configured to provide a −3.0 diopter correction at the central wavelength and to induce a counter-acting chromatic aberration of 0.0063 diopters/nm. The first example chromatic aberration correcting contact lens can be configured using FIG. 6 and FIG. 7 to have an exterior shape that provides a −6.12 diopter power (produces 0.0008 diopter/nm chromatic correction) combined with a subsurface diffractive optical element that provides 3.12 diopter power (produces 0.0057 diopter/nm) to produce a combined-3.0 diopter power and 0.0063 diopter/nm chromatic correction for correcting and achromatizing the example myopic eye of FIG. 2.

FIG. 6 shows plots 32, 34, 36, 38, 40, 42, 44, 46 of variation in optical power with wavelength for different example lenses (made of a contact lens material with an Abbe number approximately that of water (i.e. 55.74)) with different external shapes configured to provide different optical powers. The lens of plot 32 provides −7.0 diopter at the central wavelength and 0.00074 diopters/nm chromatic correction (−7.11 diopter at 400 nm, −7.0 diopter at 550 nm, and −6.89 diopter at 700 nm). The lens of plot 34 provides −5.0 diopter at the central wavelength and 0.00053 diopters/nm chromatic correction (−5.08 diopter at 400 nm, −5.0 diopter at 550 nm, and −4.92 diopter at 700 nm). The lens of plot 36 provides −3.0 diopter at the central wavelength and 0.00032 diopters/nm chromatic correction (−3.05 diopter at 400 nm, −3.0 diopter at 550 nm, and −2.95 diopter at 700 nm). The lens of plot 38 provides −1.0 diopter at the central wavelength and 0.00011 diopters/nm chromatic correction (−1.02 diopter at 400 nm, −1.0 diopter at 550 nm, and −0.98 diopter at 700 nm). The lens of plot 40 provides 1.0 diopter at the central wavelength and −0.00013 diopters/nm chromatic correction (1.02 diopter at 400 nm, 1.0 diopter at 550 nm, and 0.98 diopter at 700 nm). The lens of plot 42 provides 3.0 diopter at the central wavelength and −0.00032 diopters/nm chromatic correction (3.05 diopter at 400 nm, 3.0 diopter at 550 nm, and 2.95 diopter at 700 nm). The lens of plot 44 provides 5.0 diopter at the central wavelength and −0.00053 diopters/nm chromatic correction (5.08 diopter at 400 nm, 5.0 diopter at 550 nm, and 4.92 diopter at 700 nm). The lens of plot 46 provides 7.0 diopter at the central wavelength and −0.00074 diopters/nm chromatic correction (7.11 diopter at 400 nm, 7.0 diopter at 550 nm, and 6.89 diopter at 700 nm).

FIG. 7 shows plots 48, 50, 52, 54, 56, 58, 60, 62 of variation in optical power with wavelength for different subsurface diffractive optical structures configured to provide different optical powers. The subsurface diffractive optical structure of plot 48 provides −7.0 diopter at the central wavelength and −0.01273 diopters/nm chromatic correction (−5.09 diopter at 400 nm, −7.0 diopter at 550 nm, and −8.91 diopter at 700 nm). The subsurface diffractive optical structure of plot 50 provides −5.0 diopter at the central wavelength and −0.00909 diopters/nm chromatic correction (−3.64 diopter at 400 nm, −5.0 diopter at 550 nm, and −6.36 diopter at 700 nm). The subsurface diffractive optical structure of plot 52 provides −3.0 diopter at the central wavelength and −0.00545 diopters/nm chromatic correction (−2.18 diopter at 400 nm, −3.0 diopter at 550 nm, and −3.82 diopter at 700 nm). The subsurface diffractive optical structure of plot 54 provides −1.0 diopter at the central wavelength and −0.00182 diopters/nm chromatic correction (−0.73 diopter at 400 nm, −1.0 diopter at 550 nm, and −1.27 diopter at 700 nm). The subsurface diffractive optical structure of plot 56 provides 1.0 diopter at the central wavelength and 0.00182 diopters/nm chromatic correction (0.73 diopter at 400 nm, 1.0 diopter at 550 nm, and 1.27 diopter at 700 nm). The subsurface diffractive optical structure of plot 58 provides 3.0 diopter at the central wavelength and 0.00545 diopters/nm chromatic correction (2.18 diopter at 400 nm, 3.0 diopter at 550 nm, and 3.82 diopter at 700 nm). The subsurface diffractive optical structure of plot 60 provides 5.0 diopter at the central wavelength and 0.00909 diopters/nm chromatic correction (3.64 diopter at 400 nm, 5.0 diopter at 550 nm, and 6.36 diopter at 700 nm). The subsurface diffractive optical structure of plot 62 provides 7.0 diopter at the central wavelength and 0.01273 diopters/nm chromatic correction (5.09 diopter at 400 nm, 7.0 diopter at 550 nm, and 8.91 diopter at 700).

To achromatize and correct an example myopic eye that requires a −6.0 diopter correction, a second example chromatic aberration correcting contact lens can be configured to provide a −6.0 diopter correction at the central wavelength and to induce a counter-acting chromatic aberration to achromatize the example myopic eye. Assuming the example myopic eye provides 60.0 diopters of optical power, the chromatic aberration of the example myopic eye can be estimated using equation (3).

$${( \frac{Δ P}{Δλ} )}_{eye} = ( {P}_{d , eye} ) ( \frac{1}{{v}_{water}} ) ( \frac{1}{{λ}_{F} - {λ}_{C}} ) \\ {( \frac{Δ P}{Δλ} )}_{eye} = ( 60 diopters ) ( \frac{1}{55.74} ) ( \frac{1}{486.1 nm - 656.3 nm} ) = - \text{.0063} \frac{diopters}{nm}$$

Using FIG. 6 and FIG. 7 the second example chromatic aberration correcting contact lens can be configured to have an exterior shape that provides a −8.96 diopters of power (produces about 0.000944 diopter/nm chromatic correction) combined with a subsurface diffractive optical element that provides 2.96 diopters of power (produces 0.00538 diopter/nm) to produce a combined −6.0 diopters of power and 0.0063 diopter/nm chromatic correction for correcting and achromatizing the example hyperopic eye.

### Subsurface Diffractive Optical Structure

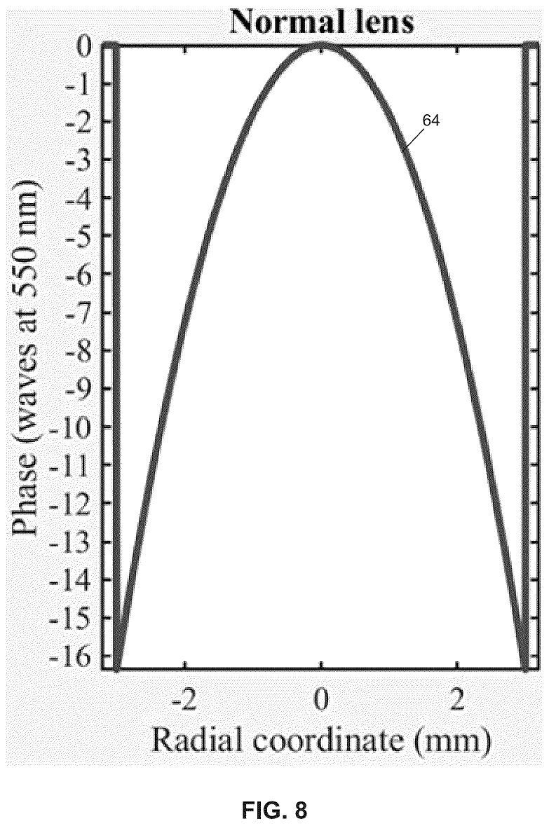

FIG. 8 illustrates a plot 64 of optical phase in waves (for a reference wavelength of 550 nm) versus radial coordinate for an example convex lens, which generates a chromatic aberration. A wavefront correction provided by a lens can be quantified using the change in optical phase in waves, as a function of spatial coordinate, provided by the lens. In geometrical optics, a wavefront is a surface with equal optical phase. Since the refractive index of a lens material is inversely proportional to the wavelength of light passing through the lens, the change in optical phase (waves) produced for each light ray passing through the lens is inversely proportional to the wavelength of light passing through the lens. For illustrative purposes, equation (1) reflects an assumed linear inverse relationship between the change in optical phase (waves) produced for each light ray passing through the lens and the wavelength of light passing through the lens. Table 1 lists changes in optical phase (waves) at different wavelengths for the example convex lens of FIG. 8 calculated using the assumed linear relationship reflected in equation (1).

$${ΔPhase}_{λ} = {ΔPhase}_{ref λ} ( 1 + {K}_{m} \frac{( λ - {λ}_{ref} )}{{λ}_{ref}} ) \qquad Equation ( 1 )$$

wherein: ΔPhase_{λ}=change in phase provided at a particular wavelength;
   ΔPhase_{refλ}=change in phase provided at the reference wavelength;
   Kₘ=material constant for change in phase for the lens material;
   λ_{ref}=the reference (design) wavelength; and
   λ=the particular wavelength

TABLE 1
Changes in optical phase at different wavelengths for the example convex lens of FIG. 8.

| 450 nm | 500 nm | 550 nm | 600 nm | 650 nm |
|---|---|---|---|---|
| 0.00 | 0.00 | 0.00 | 0.00 | 0.00 |
| −1.00 − 0.18Kₘ | −1.00 − 0.09Kₘ | −1.00 | −1.00 + 0.09Kₘ | −1.00 + 0.18Kₘ |
| −2.00 − 0.36Kₘ | −2.00 − 0.18Kₘ | −2.00 | −2.00 + 0.18Kₘ | −2.00 + 0.36Kₘ |
| −3.00 − 0.55Kₘ | −3.00 − 0.27Kₘ | −3.00 | −3.00 + 0.27Kₘ | −3.00 + 0.55Kₘ |
| −4.00 − 0.73Kₘ | −4.00 − 0.36Kₘ | −4.00 | −4.00 + 0.36Kₘ | −4.00 + 0.73Kₘ |
| −5.00 − 0.91Kₘ | −5.00 − 0.45Kₘ | −5.00 | −5.00 + 0.45Kₘ | −5.00 + 0.91Kₘ |
| −6.00 − 1.09Kₘ | −6.00 − 0.55Kₘ | −6.00 | −6.00 + 0.55Kₘ | −6.00 + 1.09Kₘ |
| −7.00 − 1.27Kₘ | −7.00 − 0.64Kₘ | −7.00 | −7.00 + 0.64Kₘ | −7.00 + 1.27Kₘ |
| −8.00 − 1.45Kₘ | −8.00 − 0.73Kₘ | −8.00 | −8.00 + 0.73Kₘ | −8.00 + 1.45Kₘ |
| −9.00 − 1.64Kₘ | −9.00 − 0.82Kₘ | −9.00 | −9.00 + 0.82Kₘ | −9.00 + 1.64Kₘ |
| −10.00 − 1.82Kₘ | −10.00 − 0.91Kₘ | −10.00 | −10.00 + 0.91Kₘ | −10.00 + 1.82Kₘ |
| −11.00 − 2.00Kₘ | −11.00 − 1.00Kₘ | −11.00 | −11.00 + 1.00Kₘ | −11.00 + 2.00Kₘ |
| −12.00 − 2.18Kₘ | −12.00 − 1.09Kₘ | −12.00 | −12.00 + 1.09Kₘ | −12.00 + 2.18Kₘ |
| −13.00 − 2.36Kₘ | −13.00 − 1.18Kₘ | −13.00 | −13.00 + 1.18Kₘ | −13.00 + 2.36Kₘ |
| −14.00 − 2.55Kₘ | −14.00 − 1.27Kₘ | −14.00 | −14.00 + 1.27Kₘ | −14.00 + 2.55Kₘ |
| −15.00 − 2.73Kₘ | −15.00 − 1.36Kₘ | −15.00 | −15.00 + 1.36Kₘ | −15.00 + 2.73Kₘ |
| −16.00 − 2.91Kₘ | −16.00 − 1.45Kₘ | −16.00 | −16.00 + 1.45Kₘ | −16.00 + 2.91Kₘ |

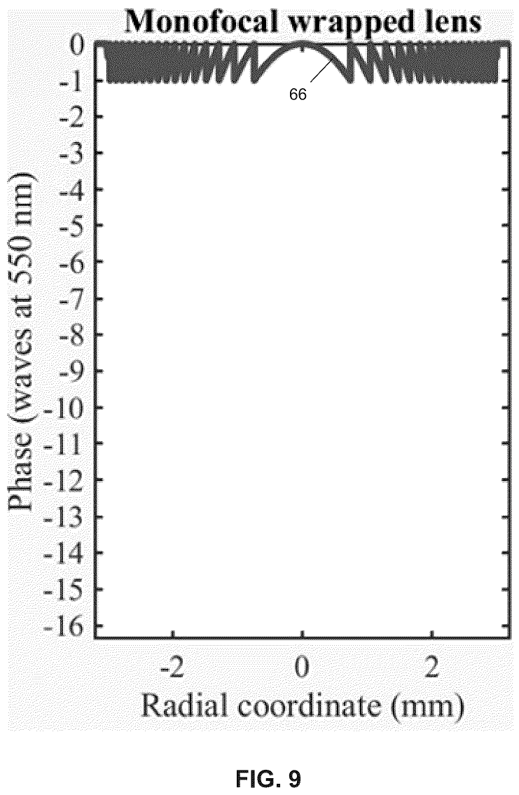

FIG. 9 illustrates a plot 66 of optical phase in waves (for a reference wavelength of 550 nm) versus radial coordinate for a phase-wrapped lens equivalent to the example lens of FIG. 8, using a phase-wrapping value of 1 wave. The phase-wrapped lens of FIG. 9 also generates a chromatic aberration.

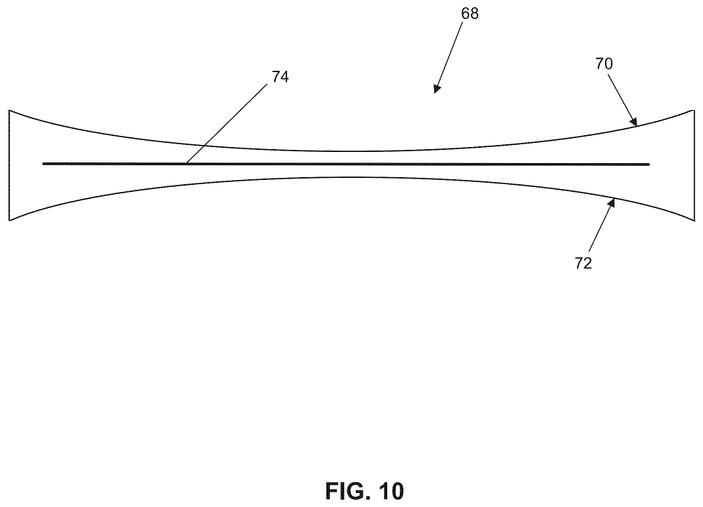

FIG. 10 shows an example chromatic aberration correcting lens 68 configured to correct a chromatic aberration of an optical component or an optical system, in accordance with embodiments. The lens 68 has external surfaces 70, 72 shaped to alter a wavefront via refraction. The lens 68 also includes at least one subsurface diffractive optical structure 74 configured alter a wavefront via refraction and diffraction. The configurations of the external surfaces 70, 72 and the at least one subsurface diffractive optical structure 74 can be selected to reduce or fully correct a chromatic aberration of an optical component or an optical system as described herein. The configurations of the external surfaces 70, 72 and the at least one subsurface diffractive optical structure 74 can also be selected to provide a suitable wavefront correction to improve image quality. The chromatic aberration correcting lens 68 can be configured as any suitable lens such as any suitable ophthalmic lens (e.g., contact lens, spectacle lens, intraocular lens) and any suitable non-ophthalmic lens. Additionally, any suitable native lens (e.g., cornea, native crystalline lens) can be modified to be configured similar to the example chromatic aberration correcting lens 68 to reduce of fully correct a chromatic aberration of the corresponding eye and can be further configured to provide a suitable wavefront correction to improve image quality.

The chromatic aberration correcting lens 68 can employ any suitable configurations of the external surfaces 70, 72 and the at least one subsurface diffractive optical structure 74. For example, as described herein, the lens 68 can be configured to correct the chromatic aberrations of the example myopic eye of FIG. 2 by having the external surfaces 70, 72 shaped to provide a −6.12 diopters of optical power and the at least one subsurface diffractive optical structure 74 configured to provide a 3.12 diopters of optical power so that the lens 68 provides a net −3.0 diopters of optical power and achromatizes the example myopic eye of FIG. 2. As another example, as described herein, the lens 68 can be configured to provide a 4.0 diopter correction and achromatize an example hyperopic eye.

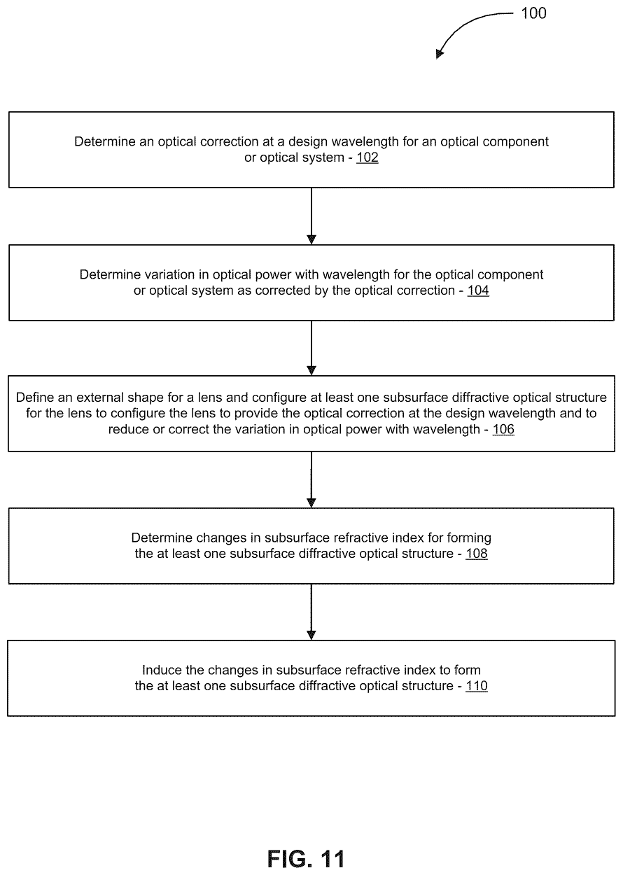

FIG. 11 show a simplified block diagram of a method 100 of configuring a lens for correcting a chromatic aberration, in accordance with embodiments. The method 100 can be configured to configure any suitable lens including, for example, the chromatic aberration correcting lens 68, which can be configured as any suitable ophthalmic lens and any suitable non-ophthalmic lens. Suitable ophthalmic lenses include, but are not limited to, natural lenses (e.g., corneas, crystalline lenses) and corrective lenses (e.g., contact lenses, intraocular lenses, spectacle lenses). The method of 100 can be practiced using any suitable ophthalmic systems including, but not limited to, the ophthalmic systems described herein.

In act 102, an optical correction at a design wavelength (e.g., at 550 nm) for an optical component or an optical system is determined. For example, an optical correction for an eye for improving image quality can be determined using any suitable approach. The determined optical correction at the design wavelength can be any optical correction that can be implemented via the combination of suitable shapes of the lens and suitable configurations of the at least one subsurface diffractive optical structures. For some optical components or optical systems, no correction at the design wavelength is desired and correction of chromatic aberrations is desired.

In act 104, the variation in optical power with wavelength (i.e., chromatic aberrations) for the corrected optical component or optical system (as corrected by the optical correction determined in act 102) is determined. Any suitable approach can be used to determine the variation in optical power with wavelength including, for example, the approaches described herein, calculation using known approaches, measuring, and/or employing known typical values such as the typical chromatic aberrations of an eye.

In act 106, an external shape for the lens is identified and the at least one subsurface diffractive optical structure for the lens is configured to provide the optical correction at the design wavelength and to reduce or correct the chromatic aberrations of the corrected optical component or optical system. As can be seen by comparing the amount of contribution to chromatic aberration correction provided by the shape of the lens (as shown in FIG. 4) with the amount of contribution to chromatic aberration correction provided by the at least one subsurface diffractive optical structure (as shown in FIG. 5), the at least one subsurface diffractive optical structure will typically provide a majority of the chromatic aberration correction for an eye. For example, in the example configuration of the lens_for correcting the chromatic aberrations shown in FIG. 2 for a myopic eye requiring −3.0 D of correction, the at least one subsurface diffractive optical structure (providing 3.12 diopter) provides about 90 percent of the chromatic aberration correction and the shape of the lens (providing −6.12 diopter) only provides about 10 percent of the chromatic aberration correction. Therefore, one approach for configuring the lens is to select a candidate configuration for the at least one subsurface diffractive optical structure that provides about 90 percent of the desired chromatic aberration correction, determine the optical power to be provided by the shape of the lens to produce the net optical correction for the lens at the design wavelength, determine the amount of chromatic aberration correction provided by the shape of the lens, and then determine the total amount of chromatic aberration correction provided by a lens having the candidate configuration of the at least one subsurface diffractive optical structure. If the total amount of chromatic aberration correction provided does not match the desired amount, iteration can be used to identify the next candidate configuration of the at least one subsurface diffractive optical structure based on whether more or less chromatic aberration correction is desired.

In act 108, the configuration of the at least one subsurface optical structure is defined including determining the associated changes to the refractive index in subsurface sub-volumes of the lens to be used to form the at least one subsurface optical structure. Any suitable approach can be used to define the configuration of the at least one subsurface optical structure. For example, in many embodiments, a wavefront map is defined that defines the wavefront changes to be induced by the at least one subsurface optical structure. The wavefront map can then be used to define the changes (including locations and amounts) in the refractive index of the lens for forming the at least one subsurface optical structure.

In act 110, the changes in the refractive index of the lens are induced to form the at least one subsurface optical structure within the lens. Any suitable approach can be used to induce the changes in the refractive index including, for example, any of the approaches described herein.

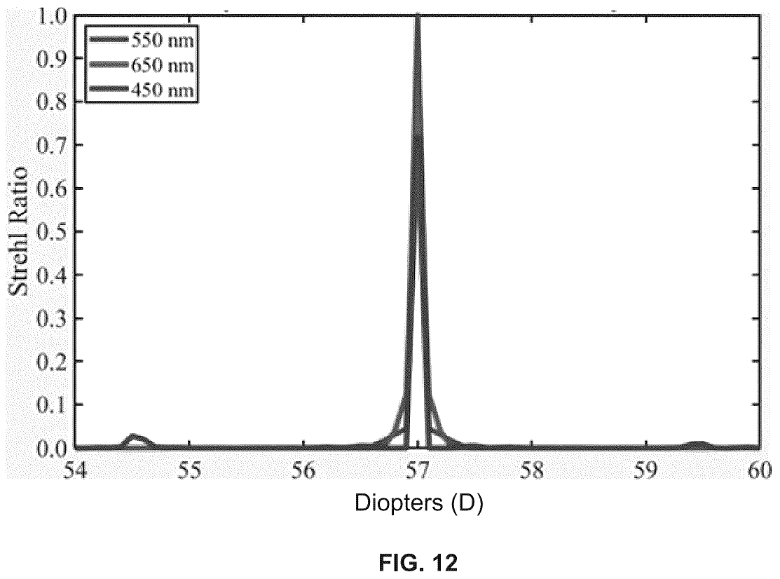

FIG. 12 shows a plot of Strehl Ratio for each of three different wavelengths (450, 550, and 650 nm) for the corrected and achromatized example myopic eye. FIG. 12 illustrates the effectiveness of utilizing the chromatic aberration correcting ophthalmic lens 68 of FIG. 10 to correct the chromatic aberrations generated by the human eye and the corrective ophthalmic lens (if any).

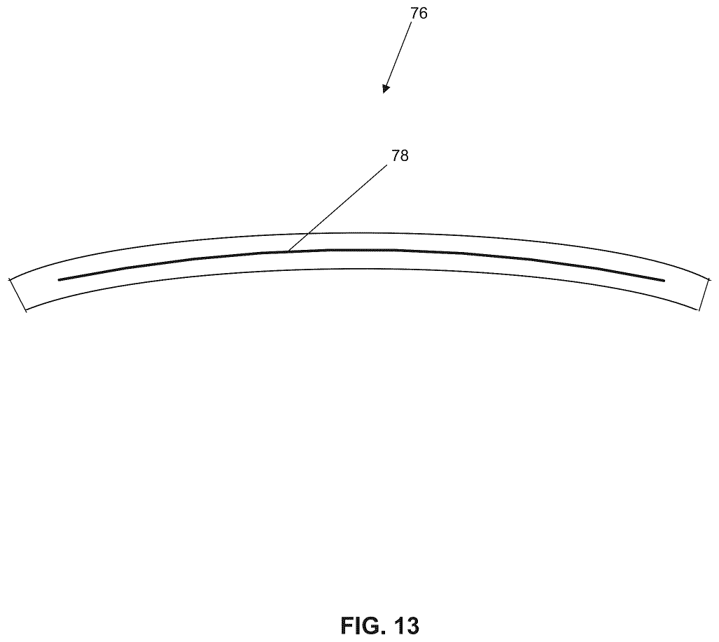

FIG. 13 shows another example chromatic aberration correcting ophthalmic lens 76 configured to correct a chromatic aberration of an optical component or an optical system, in accordance with embodiments. In the illustrated embodiment, the lens 76 is not shaped to provide any wavefront correction (except for maybe a plano wavefront correction). The lens 76 includes at least one subsurface diffractive optical structure 78 configured to alter a wavefront via refraction and diffraction. The at least one subsurface diffractive optical structure 78 can be configured (as described herein) to at least reduce and in some instances fully correct a chromatic aberration of an optical component or an optical system. The at least one subsurface diffractive optical structure 78 can also be configured to provide a suitable wavefront correction to improve image quality. The chromatic aberration correcting lens 76 can be configured as any suitable lens such as any suitable ophthalmic lens (e.g., contact lens, spectacle lens, intraocular lens) and any suitable non-ophthalmic lens. Additionally, the at least one subsurface diffractive optical structure can be formed within any suitable native lens (e.g., cornea, native crystalline lens).

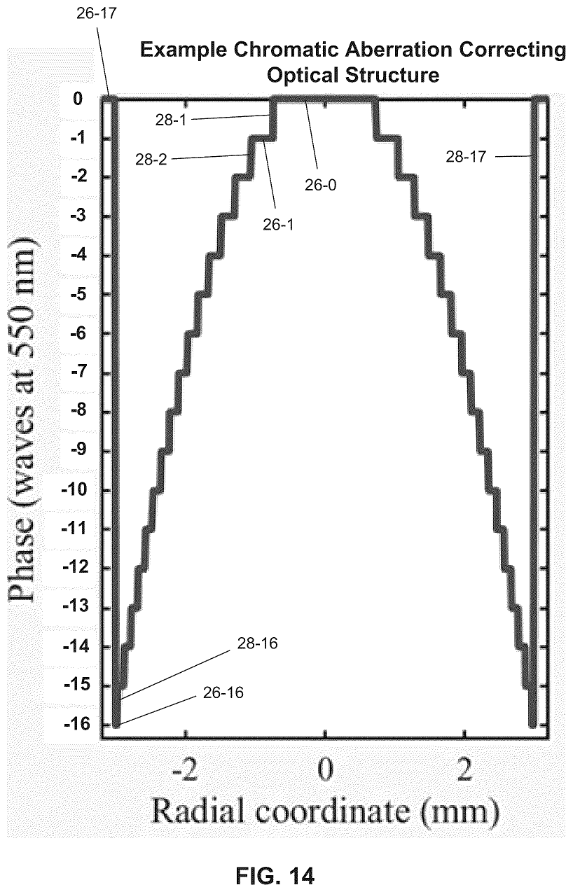

FIG. 14 illustrates a plot of optical phase in waves (for a reference wavelength of 550 nm) versus radial coordinate for a first example of the at least one subsurface diffractive optical structure 78 of the chromatic aberration correcting ophthalmic lens 76, in accordance with embodiments. The first example of the at least one subsurface diffractive optical structure 78 is configured to provide a reference wavefront correction that includes piston regions 26-0 through 26-17 of different constant whole number of optical phase in waves with respect to the reference wavelength (550 nm) and optical phase discontinuity regions 28-1 through 28-17. Each of the optical phase discontinuity regions 28-1 through 28-17 extends between and separates respective two immediately adjacent instances of the piston regions 26-0 through 26-17. The optical phase (waves) of the piston regions 26-0 through 26-17 also varies from 0.0 on the optical axis down to a negative 16.0 waves near the perimeter.

The first example of the at least one subsurface diffractive optical structure 76 provides a respective chromatic aberration correction for wavelengths that differ from the reference wavelength. The plot of FIG. 14 shows changes in optical phase in waves for the reference wavelength (550 nm) for which the piston regions 26-0 through 26-17 provide change in optical phase in waves equal to different constant whole number optical phase in waves. As a result, the piston regions 26-0 through 26-17 are invisible to the reference wavelength (550 nm). In many embodiments, the at least one subsurface diffractive optical structure 78 can be implemented by inducing suitable refractive index subsurface changes in a lens using any suitable approach such as those described herein.

In a corresponding physical embodiment of the example at least one subsurface diffractive optical structure 78 of FIG. 14, each of the piston regions 26-0 through 26-17 are not invisible to wavelengths other than the reference wavelength. In a corresponding physical embodiment, the optical phase (waves) of each of the piston regions 26-0 and 26-17 varies as a function of wavelength. To illustrate, Table 2 lists changes in optical phase (waves) provided by each of the piston regions 26-0 through 26-17 for the reference wavelength (550 nm) and four other wavelengths (450, 500, 600, and 650 nm) calculated using the assumed linear relationship reflected in equation (1). Equation (1) shows that the optical phase (waves) provided by each of the piston regions 26-0 through 26-17 is larger (in magnitude) for the shorter example wavelengths (450, 500 nm) than for the reference wavelength (550 nm) and smaller (in magnitude) for the longer example wavelengths (600, 650 nm) than for the reference wavelength (550 nm) to reduce the chromatic aberration induced by the lens and/or the eye. Table 3 lists changes in optical phase (waves) provided by each of the piston regions 26-0 through 26-17 relative to the changes in optical phase (waves) provided by each of the piston regions 26-0 through 26-17 for the reference wavelength (550 nm).

TABLE 2
Changes in optical phase (waves) for different wavelengths
provided by each of the piston regions 26-0 through 26-17.

| Region | 450 nm | 500 nm | 550 nm | 600 nm | 650 nm |
|---|---|---|---|---|---|
| 26-0 | 0.00 | 0.00 | 0.00 | 0.00 | 0.00 |
| 26-1 | −1.0 − 0.18Kₘ | −1.0 − 0.09Kₘ | −1.00 | −1.0 + 0.09Kₘ | −1.0 + 0.18Kₘ |
| 26-2 | −2.0 − 0.36Kₘ | −2.0 − 0.18Kₘ | −2.00 | −2.0 + 0.18Kₘ | −2.0 + 0.36Kₘ |
| 26-3 | −3.0 − 0.55Kₘ | −3.0 − 0.27Kₘ | −3.00 | −3.0 + 0.27Kₘ | −3.0 + 0.55Kₘ |
| 26-4 | −4.0 − 0.73Kₘ | −4.0 − 0.36Kₘ | −4.00 | −4.0+ 0.36Kₘ | −4.0 + 0.73Kₘ |
| 26-5 | −5.0 − 0.91Kₘ | −5.0 − 0.45Kₘ | −5.00 | −5.0 + 0.45Kₘ | −5.0 + 0.91Kₘ |
| 26-6 | −6.0 − 1.09Kₘ | −6.0 − 0.55Kₘ | −6.00 | −6.0 + 0.55Kₘ | −6.0 + 1.09Kₘ |
| 26-7 | −7.0 − 1.27Kₘ | −7.0 − 0.64Kₘ | −7.00 | −7.0 + 0.64Kₘ | −7.0 + 1.27Kₘ |
| 26-8 | −8.0 − 1.45Kₘ | −8.0 − 0.73Kₘ | −8.00 | −8.0 + 0.73Kₘ | −8.0 + 1.45Kₘ |
| 26-9 | −9.0 − 1.64Kₘ | −9.0 − 0.82Kₘ | −9.00 | −9.0 + 0.82Kₘ | −9.0 + 1.64Kₘ |
| 26-10 | −10.0 − 1.82Kₘ | −10.0 − 0.91Kₘ | −10.00 | −10.0 + 0.91Kₘ | −10.0 + 1.82Kₘ |
| 26-11 | −11.0 − 2.00Kₘ | −11.0 − 1.00Kₘ | −11.00 | −11.0 + 1.00Kₘ | −11.0 + 2.00Kₘ |
| 26-12 | −12.0 − 2.18Kₘ | −12.0 − 1.09Kₘ | −12.00 | −12.0 + 1.09Kₘ | −12.0 + 2.18Kₘ |
| 26-13 | −13.0 − 2.36Kₘ | −13.0 − 1.18Kₘ | −13.00 | −13.0 + 1.18Kₘ | −13.0 + 2.36Kₘ |
| 26-14 | −14.0 − 2.55Kₘ | −14.0 − 1.27Kₘ | −14.00 | −14.0 + 1.27Kₘ | −14.0 + 2.55Kₘ |
| 26-15 | −15.0 − 2.73Kₘ | −15.0 − 1.36Kₘ | −15.00 | −15.0 + 1.36Kₘ | −15.0 + 2.73Kₘ |
| 26-16 | −16.0 − 2.91Kₘ | −16.0 − 1.45Kₘ | −16.00 | −16.0 + 1.45Kₘ | −16.0 + 2.91Kₘ |

TABLE 3
Changes in optical phase (waves) for different wavelengths provided
by each of the piston regions 26-0 through 26-17 relative to the
changes in optical phase (waves) provided by each of the piston regions
26-0 through 26-17 for the reference wavelength (550 nm).

| Region | 450 nm | 500 nm | 550 nm | 600 nm | 650 nm |
|---|---|---|---|---|---|
| 26-0 | 0.00 | 0.00 | 0.00 | 0.00 | 0.00 |
| 26-1 | −0.18Kₘ | −0.09Kₘ | 0.00 | 0.09Kₘ | 0.18Kₘ |
| 26-2 | −0.36Kₘ | −0.18Kₘ | 0.00 | 0.18Kₘ | 0.36Kₘ |
| 26-3 | −0.55Kₘ | −0.27Kₘ | 0.00 | 0.27Kₘ | 0.55Kₘ |
| 26-4 | −0.73Kₘ | −0.36Kₘ | 0.00 | 0.36Kₘ | 0.73Kₘ |
| 26-5 | −0.91Kₘ | −0.45Kₘ | 0.00 | 0.45Kₘ | 0.91Kₘ |
| 26-6 | −1.09Kₘ | −0.55Kₘ | 0.00 | 0.55Kₘ | 1.09Kₘ |
| 26-7 | −1.27Kₘ | −0.64Kₘ | 0.00 | 0.64Kₘ | 1.27Kₘ |
| 26-8 | −1.45Kₘ | −0.73Kₘ | 0.00 | 0.73Kₘ | 1.45Kₘ |
| 26-9 | −1.64Kₘ | −0.82Kₘ | 0.00 | 0.82Kₘ | 1.64Kₘ |
| 26-10 | −1.82Kₘ | −0.91Kₘ | 0.00 | 0.91Kₘ | 1.82Kₘ |
| 26-11 | −2.00Kₘ | −1.00Kₘ | 0.00 | 1.00Kₘ | 2.00Kₘ |
| 26-12 | −2.18Kₘ | −1.09Kₘ | 0.00 | 1.09Kₘ | 2.18Kₘ |
| 26-13 | −2.36Kₘ | −1.18Kₘ | 0.00 | 1.18Kₘ | 2.36Kₘ |
| 26-14 | −2.55Kₘ | −1.27Kₘ | 0.00 | 1.27Kₘ | 2.55Kₘ |
| 26-15 | −2.73Kₘ | −1.36Kₘ | 0.00 | 1.36Kₘ | 2.73Kₘ |
| 26-16 | −2.91Kₘ | −1.45Kₘ | 0.00 | 1.45Kₘ | 2.91Kₘ |

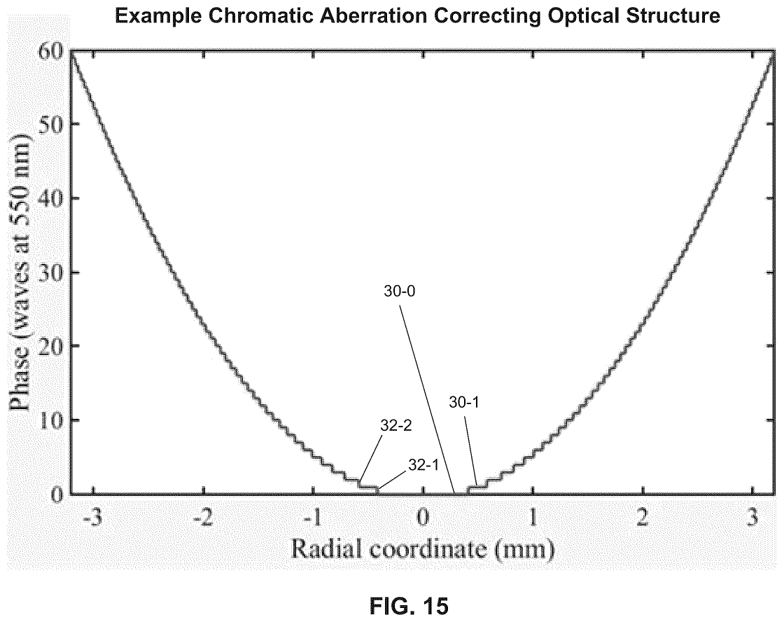

FIG. 15 illustrates a plot of optical phase (waves) for a reference wavelength (550 nm) versus radial coordinate for another example of the at least one subsurface diffractive optical structure 78, in accordance with embodiments. The example of the at least one subsurface diffractive optical structure 78 of FIG. 15 is configured similar to the first example of the at least one subsurface diffractive optical structure 78 of FIG. 14 and provides a reference wavefront correction that includes piston regions 30-0 through 30-60 of different constant whole number optical phase in waves with respect to the reference wavelength (550 nm) and optical phase discontinuity regions 32-1 through 32-60. Each of the optical phase discontinuity regions 32-1 through 32-60 extends between and separates respective two immediately adjacent instances of the piston regions 30-0 through 30-60.

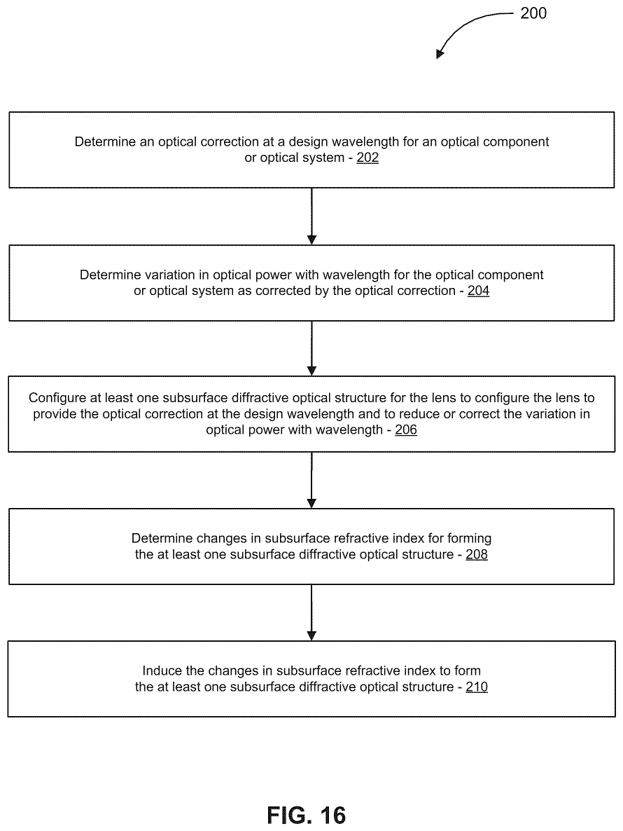

FIG. 16 show a simplified block diagram of a method 200 of configuring a lens for correcting a chromatic aberration, in accordance with embodiments. The method 200 can be configured to configure any suitable lens including, for example, the chromatic aberration correcting lens 76, which can be configured as any suitable ophthalmic lens and any suitable non-ophthalmic lens. Suitable ophthalmic lenses include, but are not limited to, natural lenses (e.g., corneas, crystalline lenses) and corrective lenses (e.g., contact lenses, intraocular lenses, spectacle lenses). The method of 200 can be practiced using any suitable ophthalmic systems including, but not limited to, the ophthalmic systems described herein.

In act 202, an optical correction at a design wavelength (e.g., at 550 nm) for an optical component or an optical system is determined. For example, an optical correction for an eye for improving image quality can be determined using any suitable approach. The determined optical correction at the design wavelength can be any optical correction that can be implemented via suitable configurations of the at least one subsurface diffractive optical structure 78 of the lens 76. For some optical components or optical systems, no correction at the design wavelength is desired and correction of chromatic aberrations is desired.

In act 204, the variation in optical power with wavelength (i.e., chromatic aberrations) for the corrected optical component or optical system (as corrected by the optical correction determined in act 202) is determined. Any suitable approach can be used to determine the variation in optical power with wavelength including, for example, the approaches described herein, calculation using known approaches, measuring, and/or employing known typical values such as the typical chromatic aberrations of an eye.

In act 206, the at least one subsurface diffractive optical structure 78 for the lens 76 is configured to provide the optical correction at the design wavelength and to reduce or correct the chromatic aberrations of the corrected optical component or optical system. The at least one subsurface diffractive optical structure 78 can have any suitable configuration, such as any suitable configuration and/or combination of the subsurface diffractive optical structures described herein.

In act 208, the configuration of the at least one subsurface optical structure 78 is defined including determining the associated changes to the refractive index in subsurface sub-volumes of the lens to be used to form the at least one subsurface optical structure. Any suitable approach can be used to define the configuration of the at least one subsurface optical structure. For example, in many embodiments, a wavefront map is defined that defines the wavefront changes to be induced by the at least one subsurface optical structure 78. The wavefront map can then be used to define the changes (including locations and amounts) in the refractive index of the lens for forming the at least one subsurface optical structure 78.

In act 210, the changes in the refractive index of the lens are induced to form the at least one subsurface optical structure 78 within the lens 76. Any suitable approach can be used to induce the changes in the refractive index including, for example, any of the approaches described herein.

The chromatic aberration correcting lenses described herein can be employed in any suitable application, including, but not limited to, ophthalmic applications. In a human eye, the main refractive components are the cornea and the natural crystalline lens. Since the refractive index of cornea varies with wavelength, the cornea generates a corresponding chromatic aberration. Likewise, since the refractive index of the natural crystalline lens varies with wavelength, the natural crystalline lens generated a corresponding chromatic aberration.

The chromatic aberration correcting subsurface diffractive optical structures described herein can be configured to reduce and, in some cases, correct chromatic aberrations induced by any suitable optical component or system. The chromatic aberration correcting subsurface diffractive optical structures described herein can further be configured to also provide a wavefront correction for improving image quality.

The chromatic aberration correcting subsurface diffractive optical structures described herein can be defined using the following approach. First, the slope of the variation of combined optical power with wavelength can be calculated for use in configuring the subsurface chromatic aberration correcting optical structure to provide a suitable counteracting chromatic aberration. As illustrated in FIG. 4, the optical power of the myopic eye corrected with a diffractive monofocal contact lens varies from 58.75 (D) at 400 nm wavelength down to 55.21 (D) at 700 nm wavelength. The slope of the variation of combined optical power with wavelength for the example corrected myopic eye of FIG. 4 is calculated in equation (2) below.

$${( \frac{Δ P}{Δλ} )}_{system} = ( \frac{55.21 D - 58.75 D}{700 nm - 400 nm} ) = - 0.0118 \frac{D}{nm} \qquad Equation ( 2 )$$

The slope of the variation of combined optical power with wavelength for the optical system composed of the myopic eye of FIG. 2 corrected with a diffractive monofocal contact lens of −3.0 D at the design wavelength of FIG. 3 can also be calculated using equation (3) set forth below.

$${( \frac{Δ P}{Δλ} )}_{system} = {( \frac{Δ P}{Δλ} )}_{eye} + {( \frac{Δ P}{Δλ} )}_{Contact lens} \\ {( \frac{Δ P}{Δλ} )}_{system} = ( {P}_{d , eye} ) ( \frac{1}{{v}_{eye}} ) ( \frac{1}{{λ}_{F} - {λ}_{c}} ) + ( \frac{{P}_{Central wavelength , Monofocal}}{{λ}_{central}} ) \\ With : \\ {λ}_{central} = 550 nm \\ {λ}_{d} = 587.6 nm \\ {λ}_{F} = 486.1 nm \\ {λ}_{C} = 656.3 nm \\ {v}_{eye} = Eye Abbe ’ s number = 55.74 \\ {( \frac{Δ P}{Δλ} )}_{system} = ( 60 D ) ( \frac{1}{55.74} ) ( \frac{1}{486.1 nm - 656.3 nm} ) + ( \frac{- 3 D}{550 nm} ) \\ {( \frac{Δ P}{Δλ} )}_{system} = - 0.0118 \frac{D}{nm} \qquad Equation ( 3 )$$

An equivalent power of a chromatic aberration correcting subsurface diffractive optical structure for achromatizing the optical system composed of the myopic eye of FIG. 2 corrected with a diffractive contact lens of −3.0 D at the design wavelength of FIG. 3 can be calculated in equation (4) below. This chromatic aberration correcting subsurface diffractive optical structure would be the result of subtracting the phase-wrapped optical structure (phase-wrapped at a value of 1 wave) with the dioptric power calculated in equation (4) below from the continuous phase optical structure with the dioptric power calculated in equation (4) below.

$$P = {( \frac{Δ P}{Δλ} )}_{system} * {λ}_{central} \\ P = {( - 0.0118 \frac{D}{nm} )}_{system} * ( 550 nm ) = - 6.4785 D \qquad Equation ( 4 )$$

FIG. 15 illustrates a plot of optical phase in waves (for a reference wavelength (550 nm)) versus radial coordinate for a chromatic aberration correcting subsurface diffractive optical structure that achromatizes the optical system composed of the myopic eye of FIG. 2 corrected with a diffractive contact lens of −3.0 D at the design wavelength of FIG. 3.

### Subsurface Diffractive Optical Structures Configured for Partial Correction of Chromatic Aberrations

In a corresponding physical embodiment of the example chromatic aberration correcting subsurface diffractive optical structure of FIG. 14, each of the piston regions 26-0 through 26-17 can be implemented by a corresponding induced refractive index that provides the corresponding constant whole number of optical phase in waves with respect to a reference wavelength. In some lens materials, however, the maximum change in refractive index that may be induced in practice may be limited by practical considerations, such as expense and/or resulting optical quality. At least where the maximum change in refractive index that may be induced in practice is limited, a subsurface diffractive optical structure configured to partially correct the chromatic aberrations can be employed.

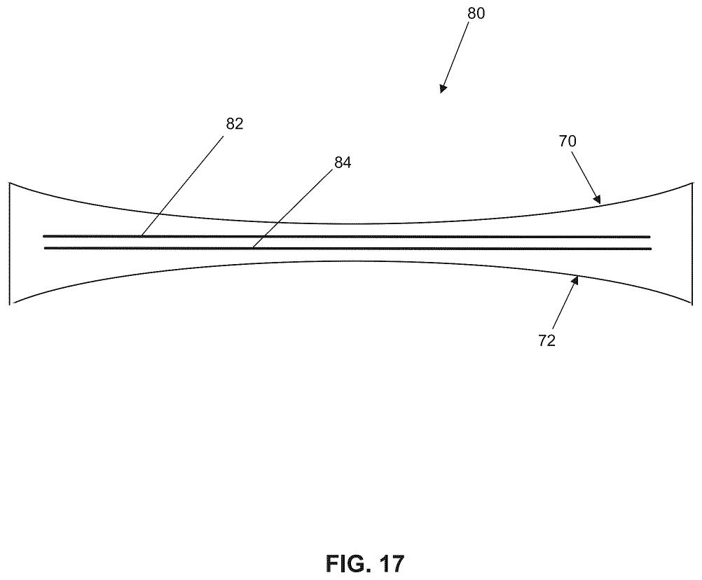

FIG. 17 shows another example chromatic aberration correcting lens 80 configured to reduce and, in some cases, correct a chromatic aberration of an optical component or an optical system, in accordance with embodiments. The lens 80 has external surfaces 70, 72 shaped to alter a wavefront via refraction. The lens 80 also includes at least one subsurface diffractive optical structure configured alter a wavefront via refraction and diffraction. The configurations of the external surfaces 70, 72 and the at least one subsurface diffractive optical structure can be selected to reduce or fully correct a chromatic aberration of an optical component or an optical system as described herein. The configurations of the external surfaces 70, 72 and the at least one subsurface diffractive optical structure can also be selected to provide a suitable wavefront correction to improve image quality. The chromatic aberration correcting lens 80 can be configured as any suitable lens such as any suitable ophthalmic lens (e.g., contact lens, spectacle lens, intraocular lens) and any suitable non-ophthalmic lens. Additionally, any suitable native lens (e.g., cornea, native crystalline lens) can be modified to be configured similar to the example chromatic aberration correcting lens to reduce of fully correct a chromatic aberration of the corresponding eye and can be further configured to provide a suitable wavefront correction to improve image quality.

In the illustrated embodiment, the at least subsurface diffractive optical structure includes a first subsurface diffractive optical structure 82 configured to induce an optical power correction and a second subsurface diffractive optical structure 84 configured to provide a chromatic aberration correction without inducing an optical power correction. The first subsurface diffractive optical structure 82 can be configured the same as the subsurface diffractive optical structure 74 to provide a combined optical power correction and chromatic aberration correction. The second subsurface diffractive optical structure 84 can be configured the same as the subsurface diffractive optical structure 78 to provide a chromatic aberration correction without inducing an optical power correction. Each of the first subsurface diffractive optical structure 82, the second subsurface optical structure 84, and the exterior surfaces 70, 72 can be configured to provide a suitable combined optical correction combined with a suitable chromatic aberration correction for an eye, or any suitable optical component or system, using the approaches described herein. The first and second subsurface diffractive optical structures 82, 84 can be combined into a single equivalent subsurface diffractive optical. The first and second subsurface diffractive optical structures 82, 84 can also be implemented in 3, 4, 5, 6, or more subsurface diffractive optical structures, which in combined effect induce the same combined wavefront correction induced by the combination of the first and second subsurface diffractive optical structures 82, 84.

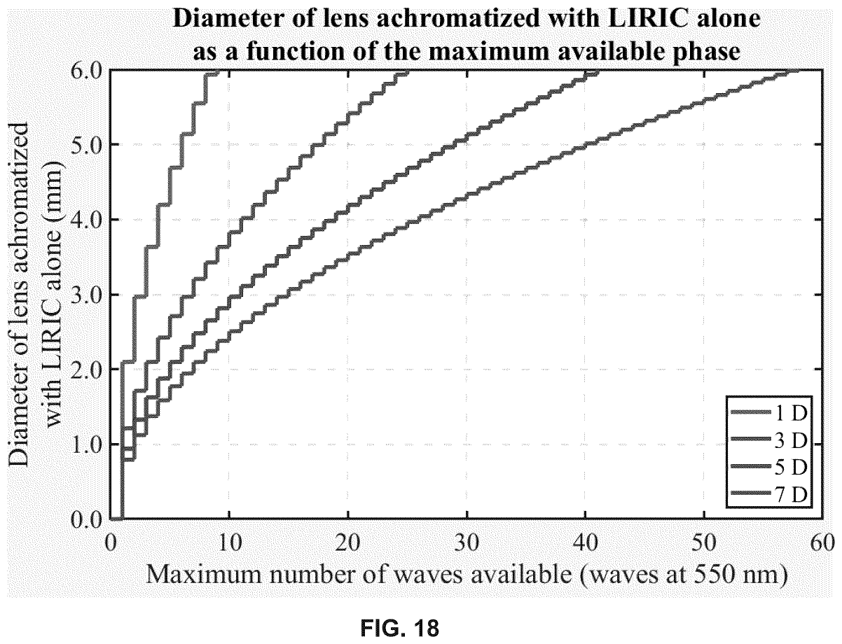

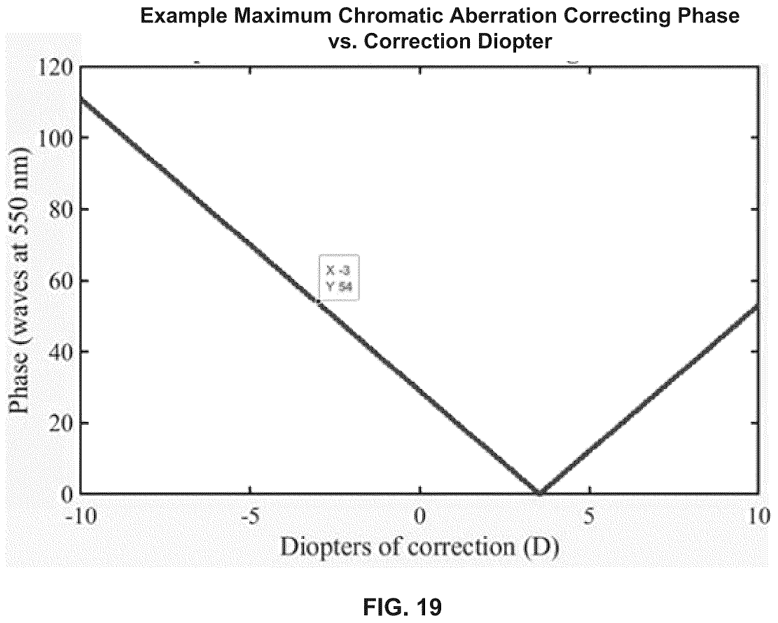

Employing the second subsurface diffractive optical structure 84 (which is configured to contribute to aberration correction without inducing an optical power correction) can be used to help deal with some potential configurational constraints. For example, for contact lenses, there may be limits on the maximum concavity of the external surfaces 70, 72 that can be employed thereby limiting the maximum amount of negative optical power that can be induced via the shape of the external surfaces 70, 74. The second subsurface diffractive optical structure 84 can be used to provide a portion of the desired chromatic aberration correction, thereby reducing the amount of concavity of the external surfaces 70, 72. On the other hand, since the amount of chromatic aberration correction that can be provided by the second subsurface diffractive optical structure 84 may be limited by the maximum amount of waves that can be employed to form the second subsurface diffractive optical structure 84, the combination of the external surfaces 70, 72 and the first subsurface diffractive optical structure can be employed to provide a portion of the desired chromatic aberration correction, thereby reducing the maximum number of waves required to form the second subsurface diffractive optical structure 84. The second subsurface diffractive optical structure 84 can also be used to enable use of a limited number of lens body configurations, for example, with exterior surfaces 70, 74 shaped to provide a limited selection of induced optical powers (e.g. −1.0 diopter, −2.0 diopter, −3.0 diopter) and still accommodate customization of the amount of chromatic aberration correction required by the lens by employing the second subsurface diffractive optical structure 84 to provide a portion of the desired total chromatic aberration correction. FIG. 18 shows plots of the maximum diameter of a chromatic aberration correcting subsurface diffractive optical structure that can be generated with a maximum phase available for several examples of equivalent powers of the chromatic aberration correcting optical structure calculated in equation (4) above. FIG. 19 shows a plot of the maximum phase necessary to create a 6 mm diameter chromatic aberration correcting subsurface diffractive optical structure as a function of the equivalent optical power in diopter of the chromatic aberration correcting optical structure calculated in equation (4) above.

### Laser and Optical Systems for Forming a Subsurface Diffractive Optical Structure.

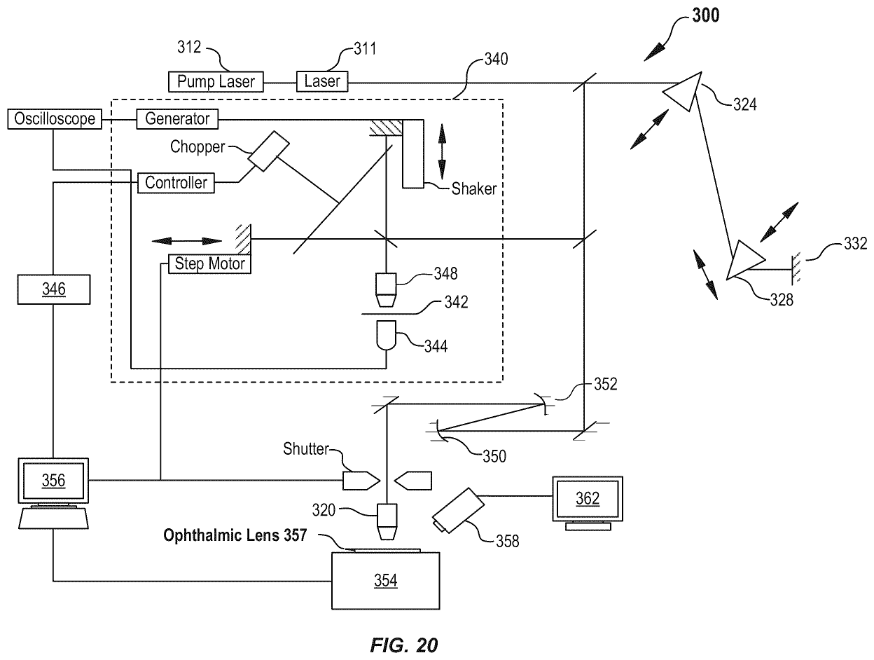

FIG. 20 is a schematic representation of the laser and optical system 300 that can be used to modify an ophthalmic lens to be configured to create high-quality vision for the patient and/or inhibit progression of myopia, in accordance with embodiments. The system 300 includes a laser source that includes a Kerr-lens mode-locked Ti:Sapphire laser 312 (Kapteyn-Mumane Labs, Boulder, Colo.) pumped by 4 W of a frequency-doubled Nd:YVO₄ laser 314. The laser generates pulses of 300 mW average power, 30 fs pulse width, and 93 MHz repetition rate at wavelength of 800 nm. Because there is a reflective power loss from the mirrors and prisms in the optical path, and in particular, from the power loss of the objective 320, the measured average laser power at the objective focus on the material is about 120 mW, which indicates the pulse energy for the femtosecond laser is about 1.3 nJ.

Due to the limited laser pulse energy at the objective focus, the pulse width can be preserved so that the pulse peak power is strong enough to exceed the nonlinear absorption threshold of the ophthalmic lens. Because a large amount of glass inside the focusing objective significantly increases the pulse width due to the positive dispersion inside of the glass, an extra-cavity, compensation scheme can be used to provide the negative dispersion that compensates for the positive dispersion introduced by the focusing objective. Two SF10 prisms 324 and 328 and one ending mirror 332 form a two-pass one-prism-pair configuration. A 37.5 cm separation distance between the prisms can be used to compensate the dispersion of the microscope objective and other optics within the optical path.

A collinear autocorrelator 340 using third-order harmonic generation is used to measure the pulse width at the objective focus. Both 2^{nd} and 3^{rd} harmonic generation have been used in autocorrelation measurements for low NA or high NA objectives. Third order surface harmonic generation (THG) autocorrelation was selected to characterize the pulse width at the focus of the high-numerical-aperture objectives because of its simplicity, high signal to noise ratio and sign of material dispersion that second harmonic generation (SHG) crystals usually introduce. The THG signal is generated at the interface of air and an ordinary cover slip 342 (Corning No. 0211 Zinc Titania glass) and measured with a photomultiplier 344 and a lock-in amplifier 346. After using a set of different high-numerical-aperture objectives and carefully adjusting the separation distance between the two prisms and the amount of glass inserted, a transform-limited 27-fs duration pulse was selected. The pulse is focused by a 60× 0.70 NA Olympus LUCPlanFLN long-working-distance objective 348.

Because the laser beam will spatially diverge after it comes out of the laser cavity, a concave mirror pair 350 and 352 is added into the optical path in order to adjust the dimension of the laser beam so that the laser beam can optimally fills the objective aperture. A 3D 100 nm resolution DC servo motor stage 354 (Newport VP-25XA linear stage) and a 2D 0.7 nm resolution piezo nanopositioning stage (PI P-622.2CD piezo stage) are controlled and programmed by a computer 356 as a scanning platform to support and locate an ophthalmic lens 357. The servo stages have a DC servomotor so they can move smoothly between adjacent steps. An optical shutter controlled by the computer with 1 ms time resolution is installed in the system to precisely control the laser exposure time. With customized computer programs, the optical shutter could be operated with the scanning stages to form the subsurface optical elements in the ophthalmic lens 357 with different scanning speed at different position and depth and different laser exposure time. In addition, a CCD camera 358 along with a monitor 362 is used beside the objective 320 to monitor the process in real time. The system 300 can be used to modify the refractive index of an ophthalmic lens to form subsurface optical elements that are configured to create high-quality vision for the patient and/or provide a myopia progression inhibiting optical correction for each of one or more locations in the peripheral retina.

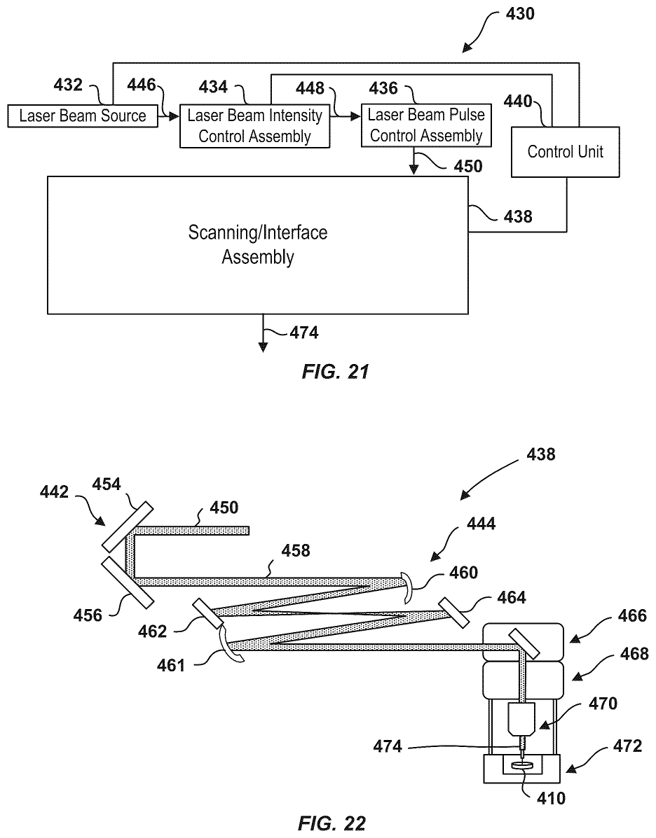

FIG. 21 is a simplified schematic illustration of another system 430 used for forming one or more subsurface optical structures within an ophthalmic lens 410, in accordance with embodiments. The system 430 includes a laser beam source 432, a laser beam intensity control assembly 434, a laser beam pulse control assembly 436, a scanning/interface assembly 438, and a control unit 440.

The laser beam source 432 generates and emits a laser beam 446 having a suitable wavelength for inducing refractive index changes in target sub-volumes of the ophthalmic lens 410. In examples described herein, the laser beam 446 has a 1035 nm wavelength. The laser beam 446, however, can have any suitable wavelength (e.g., in a range from 400 to 1100 nm) effective in inducing refractive index changes in the target sub-volumes of the ophthalmic lens 410.

The laser beam intensity control assembly 434 is controllable to selectively vary intensity of the laser beam 446 to produce a selected intensity laser beam 48 output to the laser beam pulse control assembly 436. The laser beam intensity control assembly 434 can have any suitable configuration, including any suitable existing configuration, to control the intensity of the resulting laser beam 448. In many instances, the laser beam intensity control assembly utilizes an acousto-optic modulator.

The laser beam pulse control assembly 436 is controllable to generate collimated laser beam pulses 450 having suitable duration, intensity, size, and spatial profile for inducing refractive index changes in the target sub-volumes of the ophthalmic lens 410. The laser beam pulse control assembly 436 can have any suitable configuration, including any suitable existing configuration, to control the duration of the resulting laser beam pulses 450.

The scanning/interface assembly 438 is controllable to selectively scan the laser beam pulses 450 to produce XYZ scanned laser pulses 474. The scanning/interface assembly 438 can have any suitable configuration, including any suitable existing configuration (for example, the configuration illustrated in FIG. 22) to produce the XYZ scanned laser pulses 474. The scanning/interface assembly 438 receives the laser beam pulses 450 and outputs the XYZ scanned laser pulses 474 in a manner that minimizes vignetting. The scanning/interface assembly 438 can be controlled to selectively scan each of the laser beam pulses 450 to generate XYZ scanned laser pulses 474 focused onto targeted sub-volumes of the ophthalmic lens 410 to induce the respective refractive index changes in targeted sub-volumes so as to form the one or more subsurface optical structures within an ophthalmic lens 410. In many embodiments, the scanning/interface assembly 438 is configured to restrain the position of the ophthalmic lens 410 to a suitable degree to suitably control the location of the targeted sub-volumes of the ophthalmic lens 410 relative to the scanning/interface assembly 438. In many embodiments, such as the embodiment illustrated in FIG. 22, the scanning/interface assembly 438 includes a motorized Z-stage that is controlled to selectively control the depth within the ophthalmic lens 410 to which each of the XYZ scanned laser pulses 474 is focused.

The control unit 440 is operatively coupled with each of the laser beam source 432, the laser beam intensity control assembly 434, the laser beam pulse control assembly 436, and the scanning/interface assembly 438. The control unit 440 provides coordinated control of each of the laser beam source 432, the laser beam intensity control assembly 434, the laser beam pulse control assembly 436, and the scanning/interface assembly 438 so that each of the XYZ scanned laser pulses 474 have a selected intensity and duration and are focused onto a respective selected sub-volume of the ophthalmic lens 410 to form the one or more subsurface optical structures within an ophthalmic lens 410. The control unit 440 can have any suitable configuration. For example, in some embodiments, the control unit 440 comprises one or more processors and a tangible memory device storing instructions executable by the one or more processors to cause the control unit 440 to control and coordinate operation of the of the laser beam source 432, the laser beam intensity control assembly 434, the laser beam pulse control assembly 436, and the scanning/interface assembly 438 to produce the XYZ scanned laser pulses 474, each of which is synchronized with the spatial position of the sub-volume optical structure.

FIG. 22 is a simplified schematic illustration of an embodiment of the scanning/interface assembly 438. In the illustrated embodiment, the scanning/interface assembly 438 includes an XY galvo scanning unit 442, a relay optical assembly 444, a Z stage 466, an XY stage 468, a focusing objective lens 470, and a patient interface/ophthalmic lens holder 472. The XY galvo scanning unit 438 includes XY galvo scan mirrors 454, 456. The relay optical assembly 440 includes concave mirrors 460, 461 and plane mirrors 462, 464.

The XY galvo scanning unit 442 receives the laser pulses 450 (e.g., 1035 nm wavelength collimated laser pulses) from the laser beam pulse control assembly 436. In the illustrated embodiment, the XY galvo scanning unit 442 includes a motorized X-direction scan mirror 454 and a motorized Y-direction scan mirror 456. The X-direction scan mirror 454 is controlled to selectively vary orientation of the X-direction scan mirror 454 to vary direction/position of XY scanned laser pulses 458 in an X-direction transverse to direction of propagation of the XY scanned laser pulses 458. The Y-direction scan mirror 456 is controlled to selectively vary orientation of the Y-direction scan mirror 456 to vary direction/position of the XY scanned laser pulses 458 in a Y-direction transverse to direction of propagation of the XY scanned laser pulses 458. In many embodiments, the Y-direction is substantially perpendicular to the X-direction.

The relay optical assembly 440 receives the XY scanned laser pulses 458 from the XY galvo scanning unit 442 and transfers the XY scanned laser pulses 458 to Z stage 466 in a manner that minimizes vignetting. Concave mirror 460 reflects each of the XY scanned laser pulse 458 to produce a converging laser pulses incident on plane mirror 462. Plane mirror 462 reflects the converging XY scanned laser pulse 458 towards plane mirror 464. Between the plane mirror 462 and the plane mirror 464, the XY scanned laser pulse 458 transitions from being convergent to being divergent. The divergent laser pulse 458 is reflected by plane mirror 464 onto concave mirror 461. Concave mirror 461 reflects the laser pulse 458 to produce a collimated laser pulse that is directed to the Z stage 466.

The Z stage 466 receives the XY scanned laser pulses 458 from the relay optical assembly 442. In the illustrated embodiment, the Z stage 466 and the XY stage 468 are coupled to the focusing objective lens 470 and controlled to selectively position the focusing objective lens 470 relative to the ophthalmic lens 410 for each of the XY scanned laser pulses 474 so as to focus the XYZ scanned laser pulse 474 onto a respective targeted sub-volume of the ophthalmic lens 410. The Z stage 466 is controlled to selectively control the depth within the ophthalmic lens 410 to which the laser pulse is focused (i.e., the depth of the sub-surface volume of the ophthalmic lens 410 on which the laser pulse is focused to induce a change in refractive index of the targeted sub-surface volume). The XY stage 468 is controlled in conjunction with control of the XY galvo scanning unit 442 so that the focusing objective lens 470 is suitably positioned for the respective transverse position of each of the XY scanned laser pulses 458 received by the

Z stage 466. The focusing objective lens 470 converges the laser pulse onto the targeted sub-surface volume of the lens 410. The patient interface/ophthalmic lens holder 472 restrains the ophthalmic lens 410 in a fixed position to support scanning of the laser pulses 474 by the scanning/interface assembly 438 to form the subsurface optical structures within the ophthalmic lens 410.

### Defining Subsurface Optical Elements for a Specified Optical Correction.

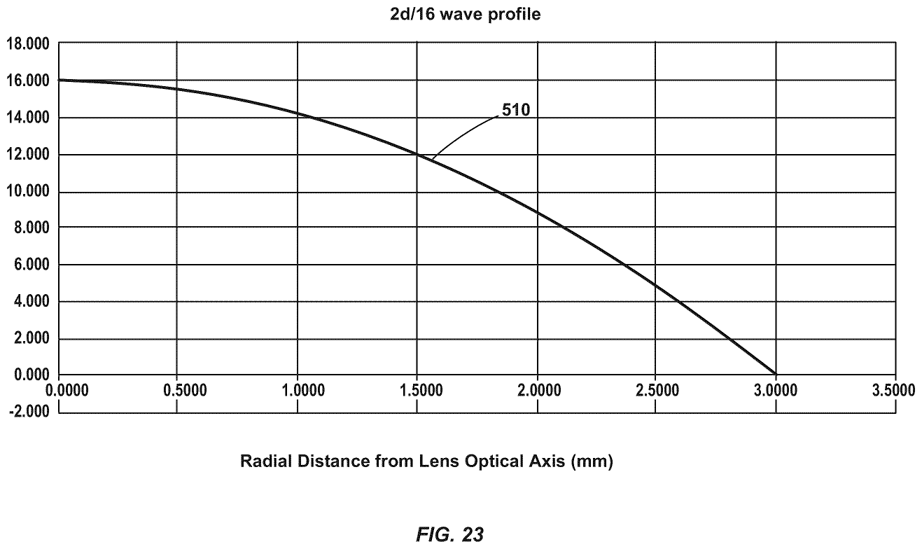

FIG. 23 through FIG. 30 illustrate a process that can be used to define subsurface optical elements for a specified optical correction. While an optical correction configured to create high-quality vision for the patient and/or inhibit progression of myopia in a subject using the approaches described herein may be a combination of any suitable number of low-order optical corrections and/or any suitable number of high-order optical corrections, a single, simple 2 diopter optical correction is illustrated. The same process, however, can be used to define subsurface optical elements for an ophthalmic lens to configure the ophthalmic lens to provide an optical correction to create high-quality vision and/or to inhibit myopia progression (by utilizing any of the myopia inhibiting optical corrections described herein).

FIG. 23 shows a radial variation in units of optical waves of a 2.0 diopter refractive index distribution 510, in accordance with embodiments. The optical waves in this curve correspond to a design wavelength of 562.5 nm. In the illustrated embodiment, the 2.0 diopter refractive index distribution 510 decreases from a maximum of 16.0 waves at the optical axis of an ophthalmic lens down to 0.0 waves at 3.0 mm from the optical axis.

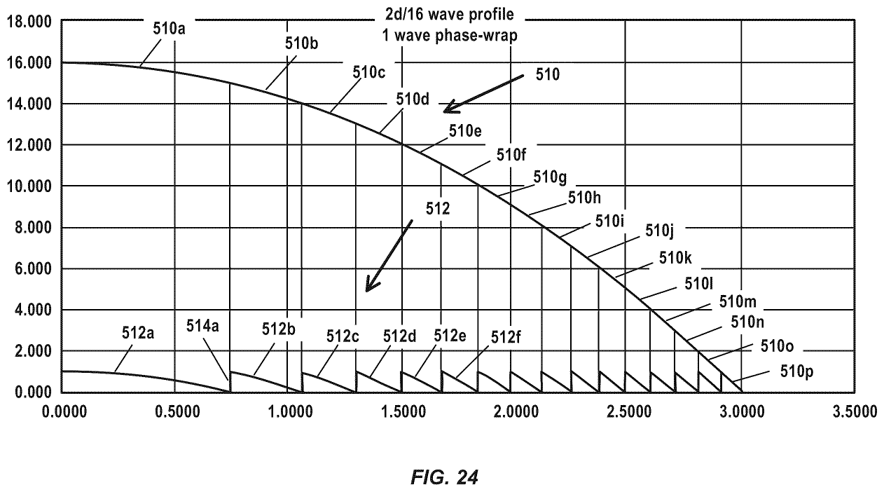

FIG. 24 shows a 1.0 wave phase-wrapped refractive index distribution 512 corresponding to the 2.0 diopter refractive index distribution 510. Each segment of the 1.0 wave phase-wrapped refractive index distribution 512 includes a sloped segment (512a through 512p). Each of all the segments, except the center segment, of the 1.0 wave phase-wrapped refractive index distribution 512 includes an optical phase discontinuity (514b through 514p) with a height equal to 1.0 wave. Each of the sloped segments (512a through 512p) is shaped to match the corresponding overlying segment (510a through 510p) of the 2.0 diopter refractive index distribution 510. For example, sloped segment 512p matches overlying segment 510p; sloped segment 5120 is equal to overlying segment 5100 minus 1.0 wave; sloped segment 512n is equal to overlying segment 510n minus 2.0 waves; sloped segment 512a is equal to overlying segment 510a minus 15.0 waves. Each sloped segment corresponds to a Fresnel zone.

The 1.0 wave height of each of the optical phase discontinuities (514b through 514p) in the distribution 512 results in diffraction at the design wavelength that provides the same 2.0 diopter refractive correction as the 2.0 diopter refractive distribution 510 while limiting maximum optical phase equal to 1.0 wave.

The 1.0 wave phase-wrapped refractive index distribution 512 requires substantially lower total laser pulse energy to induce in comparison to the 2.0 diopter refractive index distribution 510. The area under the 1.0 wave phase-wrapped refractive index distribution 512 is only about 5.2 percent of the area under the 2.0 diopter refractive index distribution 510.

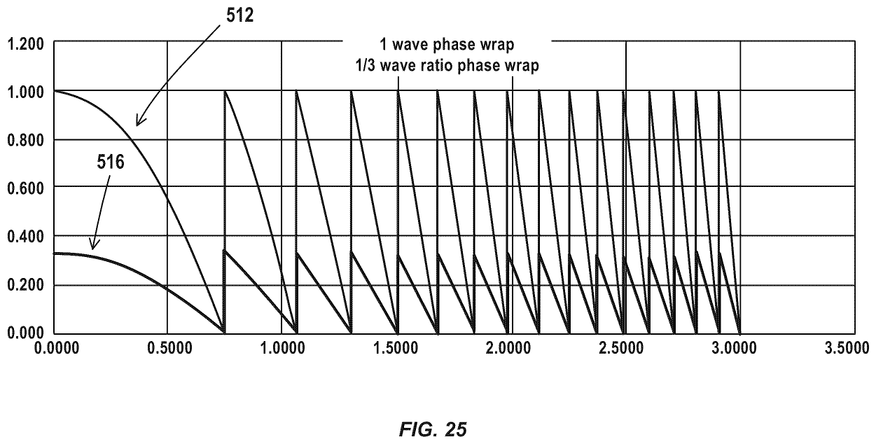

FIG. 25 shows the 1.0 wave phase-wrapped refractive index distribution 512 and an example scaled phase-wrapped refractive index distribution (for a selected maximum wave value) corresponding to the 1.0 wave phase-wrapped refractive index distribution 512. In the illustrated embodiment, the example scaled phase-wrapped refractive index distribution has a maximum wave value of ⅓ wave. Similar scaled phase-wrapped refractive index distributions can be generated for other suitable maximum wave values less than 1.0 wave (e.g., ¾ wave, ⅝ wave, ½ wave, ¼ wave, ⅙ wave). The ⅓ optical wave maximum scaled phase-wrapped refractive index distribution 516 is equal to ⅓ of the 1.0 wave phase-wrapped refractive index distribution 512. The ⅓ optical wave maximum scaled phase-wrapped refractive index distribution 516 is one substitute for the 1.0 wave phase-wrapped refractive index distribution 512 and utilizes a maximum refractive index value that provides a corresponding maximum ⅓ wave optical correction.

The ⅓ optical wave maximum scaled phase-wrapped refractive index distribution 516 requires less total laser pulse energy to induce in comparison with the 1.0 wave phase-wrapped refractive index distribution 512. The area under the ⅓ optical wave maximum scaled phase-wrapped refractive index distribution 516 is ⅓ of the area under the 1.0 wave phase-wrapped refractive index distribution 512. Three stacked layers of the ⅓ wave distribution 516 can be used to produce the same optical correction as the 1.0 wave distribution 512.

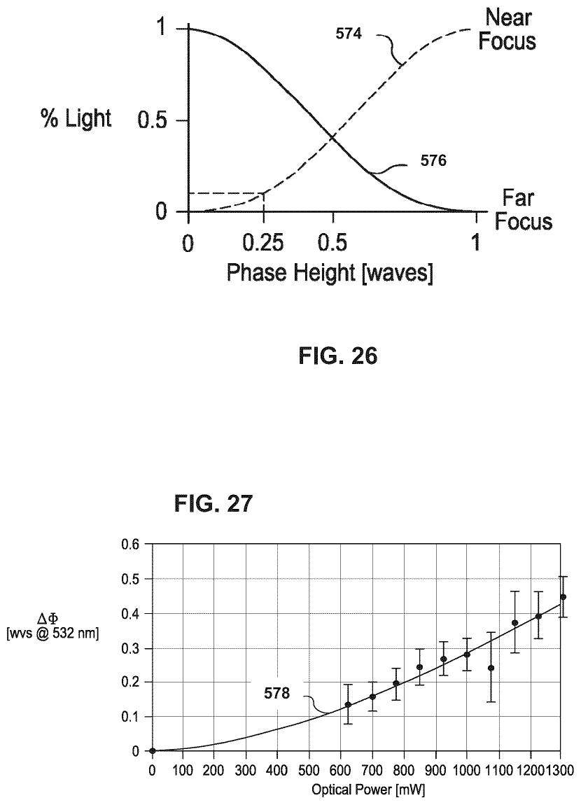

FIG. 26 graphically illustrates diffraction efficiency for near focus 574 and far focus 576 versus optical phase change height. For optical phase change heights less than 0.25 waves, the diffraction efficiency for near focus is only about 10 percent. Near focus diffraction efficiency of substantially greater than 10 percent, however, is desirable to limit the number of layers of the subsurface optical structures that are stacked to generate a desired overall optical correction. Greater optical phase change heights can be achieved by inducing greater refractive index changes in the targeted sub-volumes of the ophthalmic lens 410. Greater refractive index changes in the targeted sub-volumes of the ophthalmic lens 410 can be induced by increasing energy of the laser pulses focused onto the targeted sub-volumes of the ophthalmic lens 410.

FIG. 27 graphically illustrates an example calibration curve 578 for resulting optical phase change height as a function of laser pulse optical power. The calibration curve 578 shows correspondence between resulting optical phase change height as a function of laser average power for a corresponding laser pulse duration, laser pulse wavelength, laser pulse repetition rate, numerical aperture, material of the ophthalmic lens 410, depth of the targeted sub-volume, spacing between the targeted sub-volumes, scanning speed, and line spacing. The calibration curve 578 shows that increasing laser pulse energy results in increased optical phase change height.

Laser pulse energy, however, may be limited to avoid propagation of damage induced caused by laser pulse energy and/or heat accumulation with the ophthalmic lens 410, or even between the layers of the subsurface optical elements. In many instances, there is no observed damage during formation of the first two layers of subsurface optical elements and damage starts to occur during formation of the third layer of subsurface optical elements. To avoid such damage, the subsurface optical elements can be formed using laser pulse energy below a pulse energy threshold of the material of the ophthalmic lens 410. Using lower pulse energy, however, increases the number of layers of the subsurface optical elements required to provide the desired amount of resulting optical phase change height, thereby adding to the time required to form the total number of subsurface optical elements 412 employed.

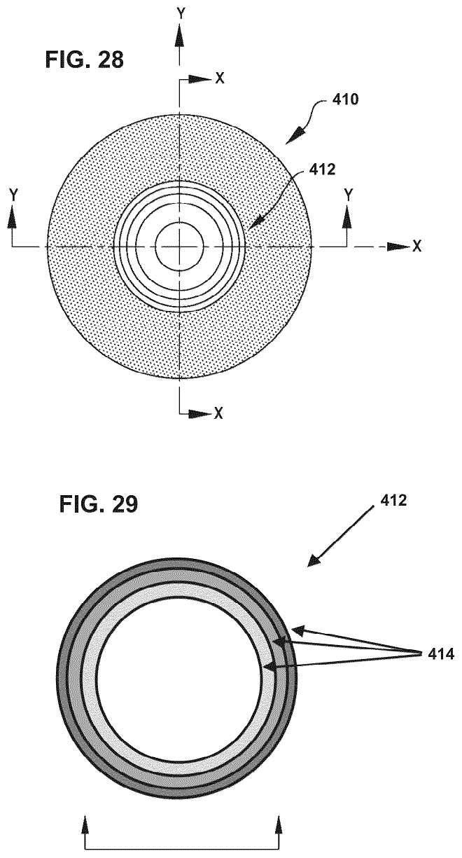

FIG. 28 is a plan view illustration of an ophthalmic lens 410 that includes one or more subsurface optical elements 412 with refractive index spatial variations, in accordance with embodiments. The one or more subsurface elements 12 described herein can be formed in any suitable type of ophthalmic lens including, but not limited to, intra-ocular lenses, contact lenses, corneas, spectacle lenses, and native lenses (e.g., a human native lens). The one or more subsurface optical elements 412 with refractive index spatial variations can be configured to provide a suitable refractive correction configured to create high-quality vision for the patient and/or inhibit progression of myopia as described herein. Additionally, the one or more subsurface optical elements 412 with refractive index spatial variations can be configured to provide a suitable refractive correction for each of many optical aberrations such as astigmatism, myopia, hyperopia, spherical aberrations, coma, and trefoil, as well as any suitable combination thereof.

FIG. 29 is a plan view illustration of one of the subsurface optical elements 412 of the ophthalmic lens 410. The illustrated subsurface optical elements 412 occupies a respective volume of the lens 410, which includes associated sub-volumes of the lens 410. In many embodiments, the volume occupied by one of the optical elements 412 includes first, second, and third portions 414. Each of the first, second, and third portions 414 can be formed by focusing suitable laser pulses inside the respective portion 414 so as to induce changes in refractive index in sub-volumes of the lens 410 that make up the respective portion 414 so that each portion 414 has a respective refractive index distribution.

In many embodiments, a refractive index distribution is defined for each portion 414 that forms the subsurface optical structures 412 so that the resulting subsurface optical structures 412 provide a desired optical correction. The refractive index distribution for each portion 414 can be used to determine parameters (e.g., laser pulse power (mW), laser pulse width (fs)) of laser pulses that are focused onto the respective portions 414 to induce the desired refractive index distributions in the portions 414.

While the portions 414 of the subsurface optical structures 412 have a circular shape in the illustrated embodiment, the portions 414 can have any suitable shape and distribution of refractive index variations. For example, a single portion 414 having an overlapping spiral shape can be employed. In general, one or more portions 414 having any suitable shapes can be distributed with intervening spaces so as to provide a desired optical correction for light incident on the subsurface optical structure 412.

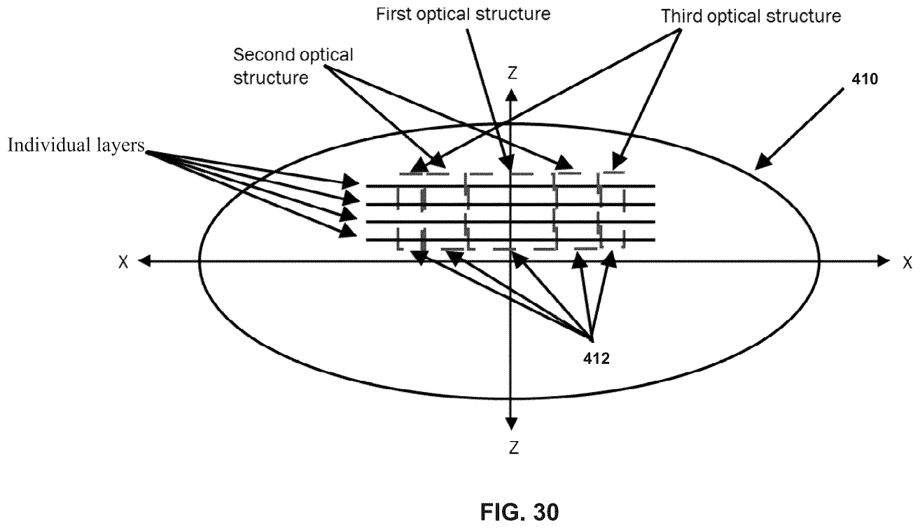

FIG. 30 illustrates an embodiment in which the subsurface optical elements 412 are comprised of several stacked layers that are separated by intervening layer spaces. In the illustrated embodiment, the subsurface optical elements 412 have a spatial distribution of refractive index variations. FIG. 30 is a side view illustration of an example distribution of refractive index variations in the subsurface optical elements 412. In the illustrated embodiment, the subsurface optical elements 412 can be formed using a raster scanning approach in which each layer is sequentially formed starting with the bottom layer and working upward. For each layer, a raster scanning approach can sequentially scan the focal position of the laser pulses along planes of constant Z-dimension while varying the Y-dimension and the X-dimension so that the resulting layers have the flat cross-sectional shapes shown in FIG. 30, which shows a cross-sectional view of the ophthalmic lens 410. In the raster scanning approach, timing of the laser pulses can be controlled to direct each laser pulse onto a targeted sub-volume of the ophthalmic lens 410 and not direct laser pulses onto non-targeted sub-volumes of the ophthalmic lens 410, which include sub-volumes of the ophthalmic lens 10 that do not form any of the subsurface optical elements 412, such as the intervening spaces between the adjacent stacked layers that can form the subsurface optical elements 412.

In the illustrated embodiment, there are three annular subsurface optical elements 412 with distributions of refractive index spatial variations. Each of the illustrated subsurface optical elements 412 has a flat layer configuration and can be comprised of one or more layers. If the subsurface optical structures are comprised of more than one layer, the layers can be separated from each other by an intervening layer spacing. Each of the layers, however, can alternatively have any other suitable general shape including, but not limited to, any suitable non-planar or planar surface. In the illustrated embodiment, each of the subsurface optical elements 412 has a circular outer boundary. Each of the subsurface optical elements 412, however, can alternatively have any other suitable outer boundary shape. Each of the subsurface optical elements 412 can include two or more separate portions 414 with each covering a portion of the subsurface optical elements 412.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1.-20. (canceled)

21. A lens comprising:
a subsurface chromatic aberration correcting optical structure, wherein each of sub-volumes of the subsurface chromatic aberration correcting optical structure has a respective refractive index spatial distribution so that the subsurface chromatic aberration correcting optical structure provides a reference wavefront correction comprising optical phase discontinuity regions and piston regions of different constant whole number optical phase in waves with respect to a reference wavelength of light, wherein each of the optical phase discontinuity regions extends between and separates respective two immediately adjacent instances of the piston regions, and wherein the subsurface chromatic aberration correcting optical structure provides a respective chromatic aberration correction for wavelengths that differ from the reference wavelength of light.

22. The lens of claim 21, wherein the subsurface chromatic aberration correcting optical structure provides no chromatic aberration correction for the reference wavelength of light.

23. The lens of claim 21, configured as an ophthalmic lens.

24. The lens of claim 23, configured as a contact lens.

25. The lens of claim 23, configured as an intraocular lens.

26. The lens of claim 23, configured as a spectacle lens.

27. The lens of claim 23, configured to produce substantially the same best-focus Strehl Ratio value for an eye at the reference wavelength of light and a second reference wavelength that differs from the reference wavelength of light by at least 50 nm.

28. The lens of claim 23, configured to result in a best-focus Strehl Ratio value occurring at the same dioptric power for all visible light wavelengths.

29. The lens of claim 23, configured to provide an optical power correction for an eye.

30. The lens of claim 29, configured to produce substantially the same best-focus Strehl Ratio value for the eye at the reference wavelength of light and a second reference wavelength that differs from the reference wavelength of light by at least 50 nm.

31. The lens of claim 29, configured to result in a best-focus Strehl Ratio value occurring at the same dioptric power for all visible light wavelengths.

32. The lens of claim 21, wherein:
the piston regions comprise a central piston region and annular piston regions that surround the central piston region; and
each of the central piston region and the annular piston regions is configured to induce a different constant whole number optical phase change in waves with respect to the reference wavelength of light.

33. The lens of claim 32, wherein the annular piston regions comprise three of the annular piston regions.

34. The lens of claim 33, wherein the annular piston regions comprise six of the annular piston regions.

35. The lens of claim 34, wherein the annular piston regions comprise nine of the annular piston regions.

36. The lens of claim 32, wherein each of the optical phase discontinuity regions provides a one optical wave discontinuity with respect to the reference wavelength of light.

37. The lens of claim 36, wherein the central piston region is configured to induce no change in waves with respect to the reference wavelength of light.

38. The lens of claim 21, comprising a lens body made of a transparent material, wherein the subsurface chromatic aberration correcting optical structure comprises portions of the lens body that have a distribution of varying refractive index.

39. The lens of claim 21 having an external surface shape configured to alter a wavefront via refraction.

40. The lens of claim 39, further comprising at least one subsurface diffractive optical structure, wherein:
the at least one subsurface diffractive optical structure comprises the subsurface chromatic aberration correcting optical structure; and
the at least one subsurface diffractive optical structure and the external surface shape are configured to jointly provide an optical power correction for an eye.
